SEQUENCE LISTING (6 sequences)

SEQ ID NO: 1 - DNA, 152 bp, synthetic construct
  source 1..152: mol_type = other DNA
ggaagccctgtacttcgatgtcacgggatgtcatatgggtcacacccagggggacgatgtccccaaagcgctgcacctcatgaatcacggcagtggtgtagggcatgtgagcctggtcacccatctctggtcgccgcacctgccctatcacg

SEQ ID NO: 2 - DNA, 152 bp, synthetic construct
  source 1..152: mol_type = other DNA
ggaagccctgtacttcgatgtcacgggatgtcatatgggtcacacccagggggacgatgtccccaaagtgctgcacctcgtgaatcacggcagtggtgcagggcatgtgagcctggtcacccatctctggtcgccgcacctgccctatcacg

SEQ ID NO: 3 - DNA, 61 bp, synthetic construct
  source 1..61: mol_type = other DNA
acccagggggacgatgtccccaaagcgctgcacctcatgaatcacggcagtggtgtagggc

SEQ ID NO: 4 - DNA, 134 bp, synthetic construct
  source 1..134: mol_type = other DNA
tgggtcacacccagggggacgatgtccccaaagcgctgcacctcatgaatcacggcagtggtgtagggcatgtgagcctggtcacccatctctggtcgccgcacctgccctatcacgtcgtcgatctcctgttg

SEQ ID NO: 5 - DNA, 134 bp, synthetic construct
  source 1..134: mol_type = other DNA
tgggtcacacccagggggacgatgtccccaaagtgctgcacctcgtgaatcacggcagtggtgcagggcatgtgagcctggtcacccatctctggtcgccgcacctgccctatcacgtcgtcgatctcctgttg

SEQ ID NO: 6 - DNA, 58 bp, synthetic construct
  source 1..58: mol_type = other DNA
gtccccaaagtgctgcacctcatgaatcacggcagtggtgtagggcatgtgagcctgg

(19) United States
(12) Patent Application Publication
(10) Pub. No.: US 2026/0139298 A1
(43) Pub. Date: May 21, 2026

(54) MULTI-REGION NUCLEIC ACID ANALYSIS

(71) Applicant: Pleno, Inc., San Diego, CA (US)

(72) Inventors: Daniel Ortiz Velez, San Diego, CA (US); Jeffrey Brodin, San Diego, CA (US)

(21) Appl. No.: 19/453,895

(22) Filed: Jan. 20, 2026

Related U.S. Application Data
(63) Continuation of application No. PCT/US2024/038497, filed on Jul. 18, 2024.
(60) Provisional application No. 63/514,320, filed on Jul. 18, 2023.

(51) Int. Cl. C12Q 1/6827; C12Q 1/34; C12Q 1/6855; C12Q 1/6888; G16B 20/20; G16B 30/00

(52) U.S. Cl. CPC C12Q 1/6827; C12Q 1/34; C12Q 1/6855; C12Q 1/6888; C12Y 301/00; G16B 20/20; G16B 30/00; C12Q 2600/154; C12Q 2600/16

(57) ABSTRACT

Provided herein are methods for detecting two or more genomic regions of interest in a target in an assay. Also provided are sets of multi-region coded recognition elements and uses thereof to detect two or more genomic regions of interest in a target.

### CROSS-REFERENCE

This application is a continuation of International Application Number PCT/US2024/038497, filed on Jul. 18, 2024, which claims the benefit of U.S.

Provisional Application Ser. No. 63/514,320, filed on Jul. 18, 2023, both of which is herein incorporated by reference in its entireties.

### INCORPORATION BY REFERENCE OF SEQUENCE LISTING

The present application is being filed along with a Sequence Listing in electronic format. The Sequence Listing is provided as a file entitled 64100_729_301_SL.xml, created Jan. 13, 2026, which is 6,911 bytes in size. The information in the electronic format of the Sequence Listing is incorporated by reference in its entirety.

### SUMMARY

Aspects disclosed herein, in some embodiments, provide a method of conducting an assay for detecting two or more genomic regions of interest (ROI) in a target of a set of targets, the method comprising: subjecting the set of targets to a recognition event, wherein each target of the set of targets is uniquely recognized by and hybridized to at least one coded recognition element from a set of coded recognition elements, wherein each coded recognition element comprises: a first target-specific binding site and a first genomic ROI binding site to a first of the two or more genomic ROI in the target of the set of targets; a second target-specific binding site and a second genomic ROI binding site to a second of the two or more genomic ROI in the target of the set of targets; and a code from a set of codes, wherein each code from the set of codes comprises at least one segment encoding one or more symbols that correspond to a sequence of one or more nucleotides, and wherein each code from the set of codes is unique for each coded recognition element from the set of coded recognition elements; subjecting the coded recognition elements from the set of coded recognition elements to a molecular transformation event to yield a set of modified coded recognition elements; and performing an amplification reaction on the modified coded recognition elements and detecting the two or more genomic ROI associated with the amplified modified coded recognition elements, and decoding the codes, thereby assaying for the two or more genomic ROI in the target of the set of targets. In some embodiments, the two or more genomic ROI are detected substantially simultaneously. In some embodiments, the set of coded recognition elements comprises at least 10 coded recognition elements and each of the coded recognition elements comprises a soft decodable code. In some embodiments, the set of coded recognition elements comprises at least 100 coded recognition elements and each of the coded recognition elements comprises a soft decodable code. In some embodiments, the set of coded recognition elements comprises at least 1,000 coded recognition elements and each of the coded recognition elements comprises a soft decodable code. In some embodiments, the set of coded recognition elements comprises at least 10,000 coded recognition elements and each of the coded recognition elements comprises a soft decodable code. In some embodiments, decoding the codes comprises: recording a signal produced in response to interrogation of each segment of the codes; and upon completion of the interrogation, determining a probability of the presence of each of the codes by applying a soft-decision probabilistic decoding algorithm to the recorded signal, wherein the presence of the code is indicative of the presence of the target. In some embodiments, interrogation of the segments comprises one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, SOLID, and sequencing by ligation. In some embodiments, the set of targets is immobilized on a surface. In some embodiments, the set of coded recognition elements is immobilized on a surface. In some embodiments, the amplification reaction is performed on a surface. In some embodiments, the amplification event and the detection event are performed on the same surface. In some embodiments, the transformation event comprises a ligation reaction to yield the set of modified coded recognition elements. In some embodiments, each of the coded recognition elements from the set of coded recognition elements comprise a 5′ probe arm and a 3′ probe arm, wherein 5′ probe arm comprises the first genomic ROI binding site and the 3′ probe arm comprises the second genomic ROI binding site of the two or more genomic ROI in the target. In some embodiments, the method further comprises a bridge element complementary to a region of the target between the first genomic ROI binding site and the second genomic ROI binding site, wherein the transformation event is possible in the presence of the two or more genomic ROI in the target, and wherein the bridge element and the coded recognition element are hybridized to the target. In some embodiments, the coded oligonucleotide probes comprise a split oligonucleotide probe or a pair of dual oligonucleotide probes, wherein one of the split or dual probes is immobilized on the surface, wherein the molecular transformation comprises a ligation reaction to yield the set of modified recognition elements each of which is a ligated encoded split oligonucleotide probe or a ligated pair of encoded dual oligonucleotide probes. In some embodiments, each coded recognition element of the set of coded recognition elements comprises one or more sequencing primer binding sites, one or more amplification primer binding sites, unique molecular identifier sequences (UMIs), one or more sample indexes, one or more restriction enzyme sites, or a combination thereof. In some embodiments, the amplification reaction yields a nanoball comprising multiple copies of the modified coded recognition element. In some embodiments, the amplification reaction comprises rolling circle amplification (RCA) to generate concatemeric amplicon products. In some embodiments, the method further comprises cleaving the concatemeric amplicon product to yield a plurality of unit length monomer fragments each comprising a copy of the code; recircularizing the unit length monomer fragments to generate recircularized monomers; and amplifying the recircularized monomers in a second RCA reaction to produce multiple RCA products of the recircularized monomers. In some embodiments, cleaving the concatemeric amplicon product to yield the plurality of unit length monomer fragments is performed with a restriction enzyme that cleaves single stranded deoxyribonucleic acid (DNA). In some embodiments, recircularizing the plurality of unit length monomer fragments comprises an end-to-end ligation reaction. In some embodiments, the method further comprises hybridizing indexed amplification primers to the unit length monomer fragments and performing a PCR reaction to produce a plurality of amplicons comprising the code and the indexed amplification primers. In some embodiments, the method further comprises subjecting the amplified modified coded recognition elements to a cleanup operation. In some embodiments, the cleanup operation comprises an exonuclease reaction to digest linear single stranded nucleic acids. In some embodiments, the amplification reaction is performed on a surface, and wherein immobilization on the surface does not comprise a protein, nucleic acid, or biotin-streptavidin based linkage to the surface. In some embodiments, the amplification reaction is performed on a surface, and wherein immobilization on the surface does not comprise a covalent attachment to the surface. In some embodiments, the surface is a charged surface. In some embodiments, the charged surface is a cation-coated surface. In some embodiments, wherein the cation-coated surface is a polylysine coated surface. In some embodiments, the amplification reaction comprises a rolling circle amplification (RCA) reaction to produce a concatemeric amplicon comprising multiple copies of the modified coded recognition element as is performed on a charged surface without a covalent attachment to the surface. In some embodiments, the amplification reaction comprises a RCA reaction to generate a concatemeric amplicon immobilized on a surface, wherein the concatemeric amplicon comprises multiple copies of the code, and wherein the surface is a charged surface, and the immobilization comprises an ionic attachment between the concatemeric amplicon and the surface. In some embodiments, the amplification reaction is a rolling circle amplification reaction and a primer for the RCA amplification reaction is supplied in solution or bound to a charged surface without a covalent attachment prior to initiation of the RCA amplification reaction. In some embodiments, the amplification reaction yields a nanoball and further comprising condensing the nanoball by addition of one or more condensing agents. In some embodiments, the condensing agent comprises one or more cationic additives. In some embodiments, the one or more cationic additives comprise one or a combination of spermidine, Mg ions, or cationic polymers. In some embodiments, the condensing agent comprises one or more multivalent oligonucleotide sequences that crosslink sites on the RCA products. In some embodiments, the condensing agent comprises inclusion of one or more modified nucleotides in the amplification reaction and further comprising crosslinking of the modified nucleotides. In some embodiments, the modified nucleotides comprise one or both of biotinylated nucleotides and nucleotides that covalently react with multifunctional linkers, wherein the crosslinking comprises inclusion of one or both of streptavidin and the multifunctional linkers. In some embodiments, the multifunctional linkers comprise one or a combination of amino nucleotides and NHS-terminated linkers. In some embodiments, the condensing agent comprises a palindrome sequence included in the RCA product. In some embodiments, the assay is conducted in vitro. In some embodiments, the assay is conducted in vitro on a surface. In some embodiments, the assay is conducted on a surface and is not performed in situ or in vivo. In some embodiments, the assay is conducted in vitro on a surface, and wherein the surface is not a fixed tissue surface. In some embodiments, the surface is a cell surface or a tissue surface. In some embodiments, the amplification reaction is not in situ or in vivo. In some embodiments, decoding the codes comprises use of soft decision decoding. In some embodiments, each of the codes in each of the coded recognition elements is the same length in nucleotides. In some embodiments, at least a subset of the codes in the set of coded recognition elements is the same length in nucleotides. In some embodiments, the codes are trellis codes and decoding the codes that are amplified comprises decoding the trellis codes. In some embodiments, decoding the codes comprises one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, and sequencing by ligation. In some embodiments, each segment of each code comprises one symbol corresponding to one or more nucleotides. In some embodiments, each code comprises up to 50 segments for a length of each code comprising up to 50 nucleotides. In some embodiments, decoding the codes comprises sequencing by synthesis (SBS). In some embodiments, each segment of each code comprises one symbol corresponding to more than one nucleotide. In some embodiments, the set of targets comprises methylated targets.

Aspects disclosed herein, in some embodiments, provide a method of conducting an assay for a set of target analytes, the method comprising: performing a recognition and amplification event on the set of target analytes present in a sample to generate a set of coded rolling circle amplification products (RCPs) from the target analytes or complements thereof, wherein each of the coded RCPs comprises: two or more copies of a code from a set of codes, wherein each code from the set of codes comprises at least one segment encoding one or more symbols that correspond to a sequence of one or more nucleotides; and two or more copies of a target nucleic acid sequence from the set of target analytes, wherein the target nucleic acid sequence comprises two or more genomic regions of interest (ROI); recording a signal produced in response to interrogation of each segment of the codes; and upon completion of the interrogation, determining a probability of the presence of each of the codes by applying a soft-decision probabilistic decoding algorithm to the recorded signal, wherein the presence of the code is indicative of the presence of the two or more genomic ROI in the target analyte. In some embodiments, the set of coded RCPs comprises at least 10, at least 100, at least 1,000, or at least 10,000 coded RCPs, and wherein each of the coded RCPs comprises a soft decodable code. In some embodiments, the two or more genomic ROI in the target analyte are determined substantially simultaneously. In some embodiments, the set of coded RCPs comprises at least 100 coded RCPs and each of the coded RCPs comprises a soft decodable code. In some embodiments, the set of coded RCPs comprises at least 1,000 coded RCPs and each of the coded RCPs comprises a soft decodable code. In some embodiments, the set of coded RCPs comprises at least 10,000 coded RCPs and each of the coded RCPs comprises a soft decodable code. In some embodiments, interrogation of the segments comprises one or a combination of nanopore sequencing, next-generation sequencing, massively parallel sequencing, Sanger sequencing, sequencing by synthesis (SBS), pyrosequencing, sequencing by hybridization, decoding by hybridization, single molecule real-time sequencing, and sequencing by ligation. In some embodiments, each segment of each code comprises one symbol corresponding to one or more nucleotides. In some embodiments, each of the codes comprises up to 50 segments, and wherein each of the codes comprises a length in nucleotides of up to 50 nucleotides. In some embodiments, interrogation of each of the segments comprises sequencing by synthesis (SBS). In some embodiments, each segment of each code comprises one symbol corresponding to more than one nucleotide. In some embodiments, each code comprises two or more, three or more, or four or more segments. In some embodiments, each code comprises three or more segments. In some embodiments, each code comprises four or more segments. In some embodiments, each code comprises five to sixteen segments. In some embodiments, interrogation of the segments comprises decoding by hybridization. In some embodiments, at least one of the segments is interrogated more than one time by hybridization with one or more hybridization probes each having at least one label to produce the signal. In some embodiments, at least four different labels are utilized in the decoding by hybridization. In some embodiments, each code comprises at least four segments and at least sixteen symbols. In some embodiments, a unique number of possibilities at each of the segments comprises up to a number of the different labels to the power of a number of the hybridizations per segment. In some embodiments, the label comprises an optical label or a fluorescent label. In some embodiments, the label comprises a fluorescent label. In some embodiments, at least one probe comprises two or more of the labels to generate a larger number of the symbols. In some embodiments, the set of targets or target analytes comprises tens, hundreds, thousands, or tens of thousands of targets or target analytes. In some embodiments, the set of targets or target analytes comprises hundreds of targets or target analytes. In some embodiments, the set of targets or target analytes comprises thousands of targets or target analytes. In some embodiments, the set of targets or target analytes comprises tens of thousands of targets or target analytes. In some embodiments, the set of targets or target analytes comprises polypeptide targets or nucleic acid targets, or a combination thereof. In some embodiments, the set of targets or target analytes comprises polypeptide targets and nucleic acid targets. In some embodiments, the set of target analytes is immobilized on a surface. In some embodiments, a set of coded recognition elements for the recognition event are immobilized on a surface. In some embodiments, the amplification event is performed on a surface. In some embodiments, the amplification event and the recognition event are performed on the same surface. In some embodiments, the assay is conducted in vitro. In some embodiments, the assay is conducted on a surface in vitro. In some embodiments, the assay is conducted on a surface and is not performed in situ or in vivo. In some embodiments, the amplification event is performed on a surface, and wherein immobilization on the surface does not comprise a protein, nucleic acid, or biotin-streptavidin based linkage to the surface. In some embodiments, the amplification event is performed on a surface, and wherein immobilization on the surface does not comprise a covalent attachment to the surface. In some embodiments, the surface is a charged surface. In some embodiments, the charged surface is a cation-coated surface. In some embodiments, the cation-coated surface is a polylysine coated surface. In some embodiments, the set of targets or target analytes is from a sample comprising one or more of whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs, or biological washes. In some embodiments, the set of targets or target analytes is from a mammalian sample or a non-mammalian sample. In some embodiments, the set of targets or target analytes is from a non-mammalian sample. In some embodiments, the sample comprises a plant sample, a viral sample, or a pathogen sample, or combinations thereof. In some embodiments, the set of targets or target analytes are for: pathogen detection; leveraging variable regions within pseudogenes to disambiguate a genotype; identifying substantially simultaneously occurring methylation events from bisulfite converted DNA or from non-treated samples; or any combination of (a) to (c). In some embodiments, the set of targets or target analytes comprises wild-type and/or mutated nucleic acid sequences. In some embodiments, the two or more genomic ROI comprise two or more point mutations, two or more substitutions, two or more insertions, two or more deletions, two or more copy number variations (CNVs), or any combination thereof. In some embodiments, the two or more genomic ROI comprise two or more substitutions, insertions and/or deletions. In some embodiments, the two or more genomic ROI comprise two or more copy number variations. In some embodiments, the set of targets or target analytes comprises extracellular DNA fragments selected for methylation patterns indicative of a cancer. In some embodiments, one or more bases of the extracellular DNA fragments are transformed prior to detection. In some embodiments, one or more bases of the extracellular DNA fragments are not transformed prior to detection. In some embodiments, the targets or target analytes comprise extracellular DNA fragments. In some embodiments, the targets or target analytes comprise extracellular DNA fragments from blood, plasma and/or serum. In some embodiments, the targets or target analytes are selected for cancer screening or diagnosis. In some embodiments, the method further comprises counting codes and estimating a quantity of the target or the target analyte based on the counts of the codes. In some embodiments, each code from the set of codes comprises a length ranging from about 3 to 100 nucleotides. In some embodiments, each code from the set of codes comprises a length ranging from about 3 to 75 nucleotides. In some embodiments, each code from the set of codes is a predetermined code. In some embodiments, each code from the set of codes is selected to avoid interaction with other assay components. In some embodiments, each code from the set of codes differs from each other code from the set of codes. In some embodiments, each code from the set of codes is homopolymer free. In some embodiments, each code from the set of codes is generated from a 4-ary nucleotide alphabet of A, C, G and T. In some embodiments, the code is generated using a 4-state encoding trellis with 3 transitions per state. In some embodiments, each code from the set of codes is generated from a 3-ary nucleotide alphabet of a set of three of A, C, G and T. In some embodiments, the code is generated using a 4-state encoding trellis with 3 transitions per state. In some embodiments, the assay is performed on a microfluidic device and wherein the set of targets or target analytes is provided in a droplet on a droplet actuator.

Aspects disclosed herein, in some embodiments, provide a system comprising a computer processor and an electrowetting cartridge, wherein the computer processor is programmed to execute any one of the methods disclosed herein.

Aspects disclosed herein, in some embodiments, provide a system for conducting an assay for a set of targets or target analytes, comprising: a reaction vessel; a reagent dispensing module; and software to execute any of the methods disclosed herein, wherein the method is executed robotically.

Aspects disclosed herein, in some embodiments, provide a set of multi-region coded recognition elements, wherein each multi-region coded recognition element comprises: a first target-specific binding site and a first genomic region of interest (ROI) binding site; a second target-specific binding site and a second genomic ROI binding site; and a code from a set of codes, wherein each code is a soft decodable code comprising at least one segment encoding one or more symbols that correspond to a sequence of one or more nucleotides. In some embodiments, the set of multi-region coded recognition elements are padlock probes. In some embodiments, the set of multi-region coded recognition elements are molecular inversion probes. In some embodiments, the set of multi-region coded recognition elements comprises at least 10 multi-region coded recognition elements. In some embodiments, the set of multi-region coded recognition elements comprises at least 100 multi-region coded recognition elements. In some embodiments, the set of multi-region coded recognition elements comprises at least 1,000 multi-region coded recognition elements. In some embodiments, the set of multi-region coded recognition elements comprises at least 10,000 multi-region coded recognition elements. In some embodiments, each multi-region coded recognition element of the set of multi-region coded recognition elements further comprises a 5′ probe arm and a 3′ probe arm, wherein 5′ probe arm comprises the first genomic ROI binding site and 3′ probe arm comprises the second genomic ROI binding site. In some embodiments, the set of multi-region coded recognition elements further comprises a bridge element that, when bound to a target, is disposed between the first genomic ROI binding site and the second genomic ROI binding site of the multi-region coded recognition element. In some embodiments, the first target-specific region of 5′ probe arm and the second target-specific region of 3′ probe arm of the multi-region coded recognition element is hybridized to the target. In some embodiments, each of the multi-region coded recognition elements in the set of multi-region coded recognition elements is a contiguous nucleic acid molecule as a result of a ligation or gap-filing ligation of 5′ probe arm, the bridge element, and 3′ probe arm.

Aspects disclosed herein, in some embodiments, provide a method for detecting two or more target fragments, the method comprising: providing: a synthetic oligonucleotide scaffold comprising a 5′ region and a 3′ region; a coded recognition element comprising: a 5′ probe arm and a 3′ probe arm, wherein 5′ probe arm has a first region complementary to the 3′ region of the synthetic oligonucleotide scaffold and 3′ probe arm has a second region complementary to a 5′ region of the target; and a soft decodable code comprising at least one segment encoding one or more symbols that correspond to a sequence of the coded recognition element; one or more bridge elements comprising a nucleic acid sequence that is complementary to a region of the synthetic oligonucleotide scaffold interposed between 5′ region and 3′ region of the coded recognition element; and introducing a sample comprising the two or more target fragments to: (i) the synthetic oligonucleotide scaffold, (ii) the coded recognition element, and (iii) the bridge element, under conditions sufficient to form a nucleic acid complex; subjecting the nucleic acid complex to a molecular transformation event in the presence of the two or more target fragments to yield a modified recognition element comprising the soft decodable code, such that the soft decodable code; and performing an amplification event of the modified recognition element comprising the soft decodable code and detecting the two or more target fragments associated with the modified recognition element by decoding the amplified soft detectable code. In some embodiments, in the presence of the two or more target fragments, the one or more bridge elements is disposed between: each of the two or more target fragments; the 3′ probe arm and one of the two or more target fragments; or 5′ probe arm and one of the two or more target fragments. In some embodiments, in the presence of the two or more target fragments, the one or more bridge elements comprises: a first bridge element disposed between each of the two or more target fragments; and a second bridge element disposed between 3′ probe arm and one of the two or more target fragments, or 5′ probe arm and one of the two or more target fragments. In some embodiments, the synthetic oligonucleotide scaffold is a splint oligonucleotide. In some embodiments, the two or more target fragments comprise cell-free DNA. In some embodiments the molecular transformation comprises ligation or gap-filling ligation between the two or more target fragments, the coded recognition element, and the bridge element to form the modified coded recognition element. In some embodiments, the modified coded recognition element comprises a circular coded recognition element. In some embodiments, the amplification event comprises rolling circle amplification (RCA).

Aspects disclosed herein, in some embodiments, provide a kit comprising: one or more coded recognition elements; one or more synthetic oligonucleotide scaffolds; one or more bridge elements; two or more target fragments; and instructions for practicing any one of the methods disclosed herein. In some embodiments, the coded recognition element comprises multi-region coded recognition elements. In some embodiments, each coded recognition element in the one or more coded recognition elements comprises (i) a 5′ probe arm and a 3′ probe arm, wherein 5′ probe arm has a first region complementary to 3′ region of the synthetic oligonucleotide scaffold and 3′ probe arm has a second region complementary to a 5′ region of the target; and (ii) a soft decodable code comprising at least one segment encoding one or more symbols that correspond to a sequence of the coded recognition element. In some embodiments, the kit further comprises one or more buffers, one or more reagents, a manual, a protocol, or any combination thereof.

Aspects disclosed herein, in some embodiments, provide a computer system for detecting two or more target fragments, the system comprising: a non-transitory memory; and a processor in communication with the non-transitory memory, the processor configured to execute the following operations in order to effectuate a method comprising the operations of: providing: (1) a synthetic oligonucleotide scaffold comprising a 5′ region and a 3′ region; (2) a coded recognition element comprising: a 5′ probe arm and a 3′ probe arm, wherein 5′ probe arm has a first region complementary to 3′ region of the synthetic oligonucleotide scaffold and 3′ probe arm has a second region complementary to a 5′ region of the target; and a soft decodable code comprising at least one segment encoding one or more symbols that correspond to a sequence of the coded recognition element (3) one or more bridge elements comprising a nucleic acid sequence that is complementary to a region of the synthetic oligonucleotide scaffold interposed between 5′ region and 3′ region of the coded recognition element; and introducing a sample comprising the two or more target fragments to: (1) the synthetic oligonucleotide scaffold, (2) the coded recognition element, and (3) the bridge element, under conditions sufficient to form a nucleic acid complex; subjecting the nucleic acid complex to a molecular transformation event in the presence of the two or more target fragments to yield a modified recognition element comprising the soft decodable code, such that the soft decodable code; and performing an amplification event of the modified recognition element comprising the soft decodable code and detecting the two or more target fragments associated with the modified recognition element by decoding the amplified soft detectable code.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 is an example of a diagram illustrating an encoding method that uses a 4-state encoding trellis with three transitions per state.

FIG. 2 is an example of a diagram illustrating an encoding trellis for a four bases per cycle pyrosequencing.

FIG. 3 is an example of a flow diagram of a non-limiting example of a targeted nucleic acid assay workflow for detecting a target site of interest.

FIG. 4 is an example of a schematic diagram illustrating a non-limiting example of a coded multi-region recognition element having a 5′ probe arm that interrogates a first genomic region of interest (ROI) (“variant 1”) and a 3′ probe arm that interrogates a second genomic ROI (“variant 2”) with a bridge element disposed between them. Ligation or gap-filling ligation may be performed between 5′ probe arm, the bridge element and 3′ probe arm when variant 1 and variant 2 are present simultaneously in a target nucleic acid.

FIG. 5A is a non-limiting example of the use of a coded multi-region recognition element to detect a target single nucleotide polymorphism (SNP) in the gene Cytochrome P450 Family 2 Subfamily D Member 6 (CYP2D6), wherein a portion of the CYP2D6 gene is shown as SEQ ID NO: 1, when a pseudogene, Cytochrome P450 Family 2 Subfamily D Member 7 (CYP2D7), wherein a portion of the CYP2D7 gene is shown as SEQ ID NO:2, may be present and comprises a high homology region in the area of the SNP of interest in the target gene. SEQ ID NO: 3 represents 5′ (left of center line) and 3′ (right of center line) arms of a coded multi-region recognition element designed to exploit three nucleotide differences between the pseudogene and the gene of interest.

FIG. 5B is an example of a schematic diagram illustrating a coded multi-region recognition element to detect a SNP of interest in CYP2D6 and not the SNP in a high homology pseudogene CYP2D7 shown in FIG. 5A, except this schematic utilizes a bridge element in combination with a coded multi-region recognition element. FIG. 6 are examples of photos (right) showing the density, size and uniformity of nanoballs generated in an RCA reaction with a schematic diagram of the corresponding coded multi-region recognition element used to generate the image (left).

FIG. 6 shows that double ligation based on a perfect match and hybridization of both the genotyping SNP and the anchor SNP to the complementary sequences in the coded multi-region recognition element yields the highest density, size and uniformity of nanoballs (top) relative to coded multi-region recognition elements and synthetic targets nucleic acid sequences having at least one off-target match for either the anchor SNP at the 3′ terminus of the recognition element (middle), or the genotyping SNP at the 5′ terminus of the recognition element (bottom).

FIG. 7 is an example of a schematic diagram illustrating a non-limiting example of a use of a coded multi-region recognition element to interrogate a first genomic ROI (“variant 1”) and a second genomic ROI (“variant 2”) simultaneously, where the first genomic ROI and the second genomic ROI variant are phased variants from a single genomic locus.

FIG. 8 is an example of a schematic diagram illustrating a non-limiting example of a use of a coded multi-region recognition element configured to perform combinatorial detection of target fragments (“fragments”) from a sample utilizing a synthetic oligonucleotide scaffold, a coded multi-region recognition element having a 3′ probe arm and a 5′ probe arm that are complementary to a 5′ region and a 3′ region of the oligonucleotide scaffold, and a plurality of bridge elements that hybridize to the oligonucleotide scaffold at regions in between the fragments. In the presence of all (in this case 3) fragments in the sample, will a ligation or gap-filling ligation take place to create the modified recognition element.

FIG. 9 shows a non-limiting example of a computing device; in this case, a device with one or more processors, memory, storage, and a network interface.

FIG. 10 shows a non-limiting example of a web/mobile application provision system; in this case, a system providing browser-based and/or native mobile user interfaces.

FIG. 11 shows a non-limiting example of a cloud-based web/mobile application provision system; in this case, a system comprising an elastically load balanced, auto-scaling web server and application server resources as well synchronously replicated databases.

### DETAILED DESCRIPTION

Many assays, such as single base detection assays, may include a high-level of sensitivity and specificity and may be associated with a low signal. Low signal may include amplification (e.g., PCR, immunostaining cascades, and the like), resulting in complex and lengthy protocols, high-level of background, and other biases limiting the performance of the assay. There is a need in the art for assays that are easier to read and detect at higher sensitivity than the analyte itself.

The inventive concepts herein relate to encoded assays, in which a target analyte is detected based on an association of the target with a code, and detection of the code as a proxy for detection of the target analyte.

Provided herein are methods of conducting an assay for simultaneously detecting two or more genomic regions of interest (ROI) in a target of a set of targets. The method may include subjecting the set of targets to a recognition event. Each target may be uniquely recognized by and hybridized to at least one coded recognition element from a set of coded recognition elements. Each coded recognition element may comprise a first target-specific binding site and a first genomic ROI binding site to one of the two or more genomic ROI in the target from the set of targets. Each coded recognition element may comprise a second target-specific binding site and a second genomic ROI binding site to a second of the two or more genomic ROI in the target from the set of targets. Each coded recognition element may comprise a code from a set of codes, wherein each code comprises at least one segment encoding one or more symbols that correspond to a sequence of one or more nucleotides and wherein each code is unique for each coded recognition element from the set of coded recognition elements. The method may include subjecting the coded recognition elements to a molecular transformation event to yield a set of modified coded recognition elements. The method may include performing an amplification reaction on the modified recognition elements and simultaneously detecting the two or more genomic ROI sequences associated with the amplified modified coded recognition elements and decoding the codes thereby assaying for the two or more genomic ROI in the target of the set of targets.

### I. METHODS

### Multi-Region Encoded Assays

The disclosure provides encoded assays for detection of target analytes in a sample. At a high level, in an encoded assay, a target analyte (“target”) is detected based on association of the target with a code and detection of the code is a proxy for detection of the analyte. In some embodiments, the encoded assay is a multi-region encoded assay when it can detect multiple genomic regions of interest (ROI) simultaneously in a single target.

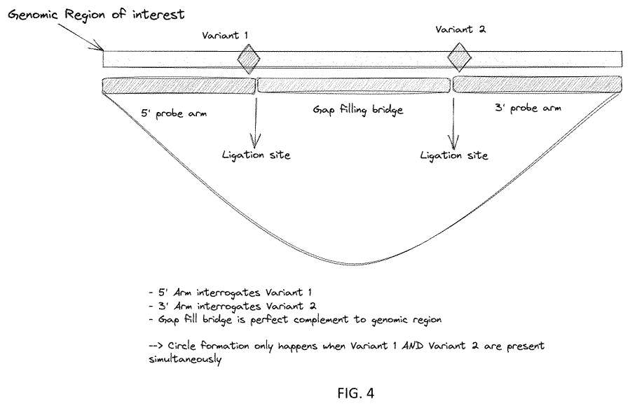

In various embodiments, an encoded assay may include a recognition event in which a target is uniquely recognized by the recognition element. The recognition event may be affected by submitting targets of a set of targets to a recognition event, in which each target is uniquely recognized by and hybridized to a recognition element associated with a code, thereby yielding a set of coded targets comprising the target and the recognition element. The recognition element may be a padlock probe, or a molecular inversion probe. The recognition element may be a multi-region recognition element, which has a 5′ probe arm and a 3′ probe arm. In some embodiments, at least one or both of 5′ probe arm and 3′ probe arm comprise a recognition motif configured to bind to two or more genomic ROIs in the target. For example, 5′ probe arm may have a binding site or recognition motif for binding a first genomic region of interest (ROI) variant, and 3′ probe arm may have a binding site or recognition motif for binding a second genomic ROI. In some embodiments, the first genomic ROI is at a different locus of the target than the second genomic ROI. In some embodiments, the recognition element, when hybridized to the target, further comprises a bridge element hybridized to the target and disposed between 5′ probe arm and 3′ probe arm. Referring to FIG. 4, the circularization formation happens when the first genomic ROI (“variant 1”) and the second genomic ROI (“variant 2”) are present in the target simultaneously.

In various embodiments, an encoded assay may include a transformation event, in which a high-fidelity molecular transformation of the recognition element associated with a code produces a modified recognition element. The transformation event may be affected by submitting each recognition element of the set of coded targets to a transformation event, in which a molecular transformation of each recognition element produces a modified recognition element, thereby yielding a set of modified recognition elements comprising the code. The transformation event is a ligation or a gap-fill ligation reaction. For example, 5′ probe arm, the bridge element and 3′ probe arm are ligated to form a modified recognition element in the form of a contiguous nucleic acid molecule.

In various embodiments, an encoded assay may include a detection event, which detects the code as a proxy for detection of the analyte, e.g., by decoding, the code, such as by detecting the code and decoding the detected code (and optionally other elements). The detection event may include an amplification operation in which each code of the set of modified recognition elements is amplified, thereby yielding a set of amplified codes. Amplified codes of the set of amplified codes may have their sequences determined or detected using a variety of techniques, including for example, but not limited to, microarray detection, nucleic acid sequencing, or detection by hybridization. In some cases, the detection operation may be integrated with the amplification operation, e.g., as in amplification with intercalating dyes.

In one embodiment, the method may include:
(i) submitting each target of a set of targets to a recognition event, in which two or more genomic ROIs of each target are simultaneously recognized by and hybridized to a recognition element associated with a code, thereby yielding a set of coded targets comprising the target and the recognition element including the code;
(ii) submitting each recognition element of the set of coded targets to a transformation event, in which a molecular transformation of each recognition element produces a modified recognition element, thereby yielding a set of modified recognition elements comprising the code;
(iii) submitting the set of modified recognition elements to an amplifying event, in which each code along with the other elements in the recognition element is amplified, thereby yielding a set of amplified codes;
(iv) submitting each amplified code of the set of amplified codes to one or more detection events, wherein the detection events are compiled and decoded thereby decoding the code.

In one embodiment, the method may include:
(i) a recognition event in which two or more genomic ROIs of the target are simultaneously recognized by a recognition element, which associates a code (and optionally other elements) with the target via the recognition element;
(ii) a transformation event, in which a high-fidelity molecular transformation of the recognition element produces a modified recognition element comprising a code;
(iii) a detection event, which detects the code as a proxy for detection of the analyte, e.g., by recognizing the presence of the code and decoding the code (and optionally other elements).

As described in more detail herein, the recognition event, transformation event, and the detection event may occur sequentially, or combinations of the operations may occur simultaneously, e.g., as a single combined operation. For example, the transformation event and the amplification event may be simultaneous, such that the sequential process involves: (i) a recognition event, followed by (ii) a transformation event/amplification event, followed by (iii) a detection event.

To further illustrate the encoded assays:
(i) In the recognition event, two or more genomic regions of interest (ROIs) of the target may be simultaneously detected by a targeted molecular binding event, such as binding of the target by a complementary sequence or a polypeptide binder.
(ii) In the transformation event, a ligation or a gap-fill ligation may produce the modified recognition element, e.g., a version of the recognition element that is ligated or gap-filled and ligated.
(iii) In the recognition event, a code reagent may be associated with the modified recognition element based on recognition of the modified recognition element. For example, the coded multi-region recognition element of the inventive concepts may be configured with a sequence that recognizes the modified recognition element and may circularize if the modified recognition element is present.
(iv) In the decoding event, the decoding of the code may involve any means of decoding the code (and optionally other elements).

The codes may be error corrected so they can be detected at low abundance and in the presence of high level of background and in the presence of many other codes.

The inventive concepts provide for multi-omic assays where a sample may be analyzed in multiple parallel workflows that are analyte-dependent, wherein converged codes can be detected simultaneously on a single platform. Parallel assay workflows may be merged into a single workflow, where multiple targets and target-types (e.g., nucleic acids and polypeptides) may be detected simultaneously in a single workflow and also read simultaneously within the same readout platform.

Following recognition and transformation, the codes may be detected, decoded and matched to targets for identification and/or quantification of targets present in the sample.

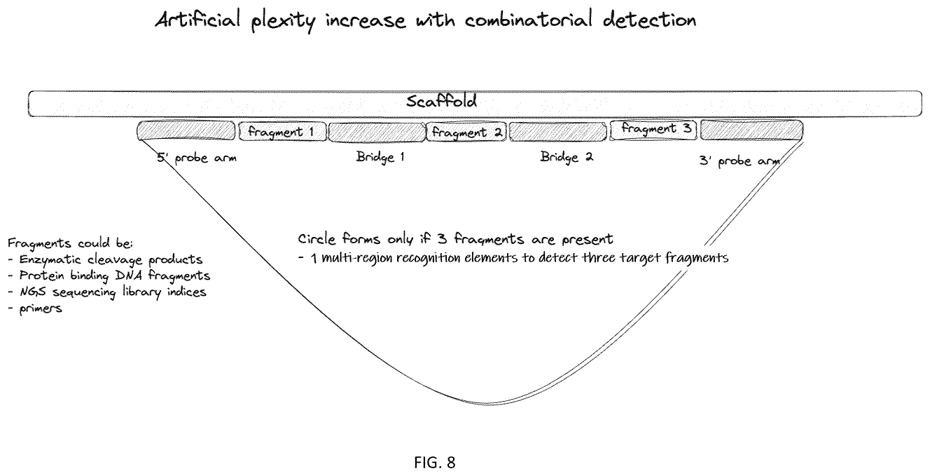

Also provided are methods for detecting two or more target fragments with a coded multi-region recognition element. In some embodiments, the methods comprise providing a synthetic oligonucleotide scaffold comprising a 5′ region and a 3′ region; a coded recognition element; and one or more bridge elements, such as those provided in FIG. 8. In some embodiments, the coded recognition element comprises a 5′ probe arm and a 3′ probe arm, wherein 5′ probe arm has a first region complementary to 3′ region of the synthetic oligonucleotide scaffold and 3′ probe arm has a second region complementary to a 5′ region of the synthetic oligonucleotide scaffold; and a code comprising at least one segment encoding one or more symbols that can be used as a proxy for the presence of the synthetic oligonucleotide scaffold. In some embodiments, the one or more bridge elements comprise a nucleic acid sequence that is complementary to a region of the synthetic oligonucleotide scaffold interposed between 5′ region and 3′ region of the recognition element.

In some embodiments, a sample potentially comprising two or more target fragments is introduced to: (i) the synthetic oligonucleotide scaffold, (ii) the coded recognition element, and (iii) the one or more bridge elements, under conditions sufficient to form a nucleic acid complex. In some embodiments, in the presence of the two or more target fragments, the one or more bridge elements may be disposed between: each of the two or more target fragments; the 3′ probe arm and one of the two or more target fragments; or 5′ probe arm and one of the two or more target fragments. Referring to FIG. 8, in the presence of the two or more target fragments, the one or more bridge elements may comprise: a first bridge element disposed between first and second target fragments; and a second bridge element disposed between second and third target fragments. A bridge element may also be located between 3′ probe arm of a recognition element and a target fragment and/or 5′ probe arm of a recognition element and a target fragment, in addition to one or more bridge elements located between one or more target molecules between 5′ and 5′ probe arms of a recognition element, or any combination thereof.

In some embodiments, the nucleic acid complex is subjected to a molecular transformation event in the presence of the two or more target fragments to yield a modified recognition element comprising the code, such that the code of the modified recognition element can be amplified with other recognition element sequences in an amplification event. In some embodiments, an amplification event of the modified recognition element is performed, thereby detecting the two or more target fragments associated with the modified recognition element, or complements thereof, by detecting and decoding the code that is amplified.

### Code Design and Decode

The encoded assays of the inventive concepts herein may make use of codewords or codes. The codes may be detected as proxies in the place of direct detection and analysis of target analytes. As an example, a target analyte may be a particular nucleic acid fragment (e.g., a nucleic acid fragment with a specific mutation). In the assays of the inventive concepts, a code may be associated with the nucleic acid fragment due to its inclusion in the recognition element that includes the code, and the code may be detected and decoded to identify the presence of the nucleic acid fragment from the sample.

For example, a code may be a predetermined sequence ranging from about 3 to about 100 nucleotides or about 3 to about 75 nucleotides. In some embodiments, the code may comprise a sequence of more than or equal to 2, 3, 4, 5, 6, 7, 8, 9, 10, 15, 20, 25, 30, 35, 40, 45, 50, 55, 60, 65, 70, 75, 80, 85, or 90 nucleotides. In some embodiments, the code may comprise a sequence of less than or equal to 90, 85, 80, 75, 70, 65, 60, 55, 50, 45, 40, 35, 30, 25, 20, 15, 10, 9, 8, 7, 6, 5, 4, 3, or 2 nucleotides. Codes may have sequences selected to avoid inadvertent interaction with other assay components, such as target sequences, other recognition element sequences, or primers. Code sequences may be selected to ensure that codes differ for each recognition element to permit unique identifiability of a target of interest during the decoding process.

### Homopolymer-Free Encoding

In one embodiment, the codes are homopolymer-free codes. For standard genomic applications that use a full 4-ary nucleotide alphabet of {ACGT}, the method uses a 4-state encoding trellis with 3 transitions per state.

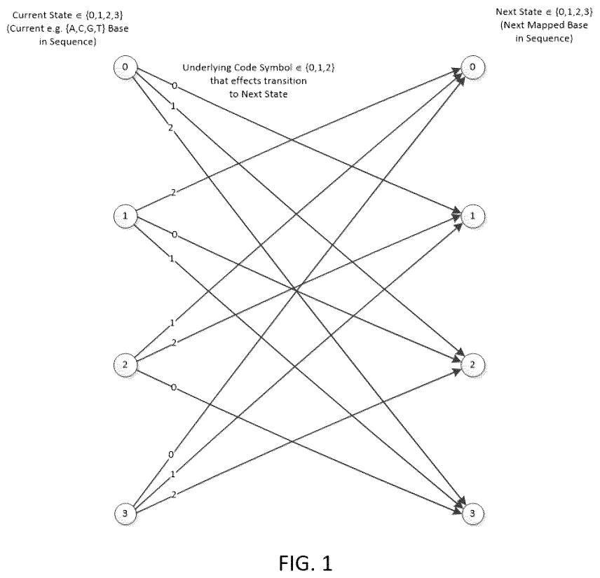

As illustrated in FIG. 1, the current state is the last mapped nucleotide, and the next state is the next (to-be) mapped nucleotide. By forbidding a transition from the current state (say, the ‘A’ state) in the present trellis section (of 4 states), to the analogous same state (of ‘A’) in the next trellis section (of 4 states), a repeated mapping to the same nucleotide base—in any generated sequence—is avoided. An ‘A’ state can transition to a ‘C’, ‘G’, or ‘T’ state in the next trellis section. Since this involves 3 transitions per state, the mapping trellis is mated to an underlying 3-ary (e.g., ternary-) alphabet error correction code that drives transitions through trellis sections. The underlying (ternary) error correction code is the mechanism that guarantees all generated codewords differ in multiple sequence positions. A similar method may apply to 3-ary alphabets (where 3 of the four nucleotide bases, say {CGT} are used), and 5-ary or higher alphabets, where the underlying correction code uses an alphabet of order one less than the mapping alphabet.

In one embodiment, codes for the set of codes are selected using a 4-ary alphabet, avoid homopolymers, and every code in the set is different from every other code in the set. The codes may be generated using the trellis method.

In one embodiment, codes for the set of codes are selected using a 3-ary alphabet, avoid homopolymers, and every code in the set is different from every other code in the set. The codes may be generated using the trellis method.
(i) In another embodiment, a homopolymer-free code composed from a 4-ary nucleotide alphabet of {ACGT} may be generated as follows:
(ii) From GF (4) (e.g., the quaternary algebraic alphabet), select an error correction code that will deliver many more codewords than necessary (because some of the generated codewords will later be eliminated);
(iii) Generate all of the codewords for the code;
(iv) Assess the number of repeated symbol locations in each codeword;
(v) Re-order the list of codewords, sorting by the number of base-repeat instances in each codeword.
(vi) From the re-ordered sort, keep the top K codewords, where K is the desired library size of codewords (this will eliminate the codewords with the highest number of polymer-repeats; each repeat may include subsequent fixing that weakens the overall code.)
(vii) For each codeword in the list of survivors, ‘smart fix’ the repeat positions in each codeword with the following procedure:
   a. Start from the beginning base position in a codeword, and find the first repeat instance of a base;
   b. Go to the second base in the first repeat instance, its base assignment may include change;
   c. If the second base is not at the end of a codeword, look ahead one base position in the codeword, and assess the assignment there;
   d. For the second base (in the repeat), choose a new base assignment that is also different from the base assigned one sample ahead; n that, in addition to removing a length-2 run, this operation will also fix a length-3 run;
   e. Process the revised codeword at each remaining repeat location, fixing the second base in each repeat using the process outlined in operations (c)-(d).

This method may eliminate all repeats. The same method can be applied to generate homopolymer code for 3-ary alphabets (e.g., {C, G, T}), and larger 5-ary+alphabets (such as oligopolymers).

Codes may be optimized for pyrosequencing and similar cyclic serial dispensation schemes. In one embodiment, the inventive concepts provide a locus code-encoding approach for pyrosequencing or similar serial (rather than pooled) primer dispensation methods. The method may generate homopolymer-free codes.

When the locus code is encapsulated between header and tail bases, all generated codes may finish decoding at the same time. The technique may avoid unexpected spurious incorporations that change how long in time that a code needs to finish being decoding. This is important because a sequencer need sample for a prescribed number of cycles to obtain complete data for decoding the samples, regardless of the underlying code. This also keeps all code candidates aligned, so that the theoretical design distances between codes are maintained.

The synchrony mentioned herein may ensure that soft decision block decoding techniques can be applied during the decoding of its blocks of samples. This soft decision decoding may guarantee that signal to noise ratio or SNR requirements are improved by at least 2 dB and sometimes by many factors-more when the signal strength significantly fades.

In pyrosequencing, nucleotides are dispensed sequentially (and non-overlappingly) in a cycle, such as G, C, T, A, G, C, T, A, G, C, . . . , etc. In some embodiments, this encoding does not directly encode bases; instead, it encodes base positions within G, C, T, A cycles. Each cycle element can be either populated, or unpopulated—and multiple elements within a cycle can be populated. For this to be implemented, the underlying code may be derived from a binary alphabet, with 1s and 0s. To emphasize, with these codes, more than one base can be incorporated within a single G, C, T, A dispensation cycle. This also implies that sequencing, though serial in nature, can be fast. And with the underlying {0,1} alphabet that underpins and drives the encoding of the populated/unpopulated cycle positions, all codes are guaranteed to be of the same length—and to finish decoding in the same amount of time.

To provide coding gain, the sequence of 0s and 1s that comprise each code may be derived from constructions of optimal binary error correction codes. Such codes possess many redundant parity bits, and these parity bits are designed such that each code varies from each other in multiple positions. This quality results in strong error correction capabilities.

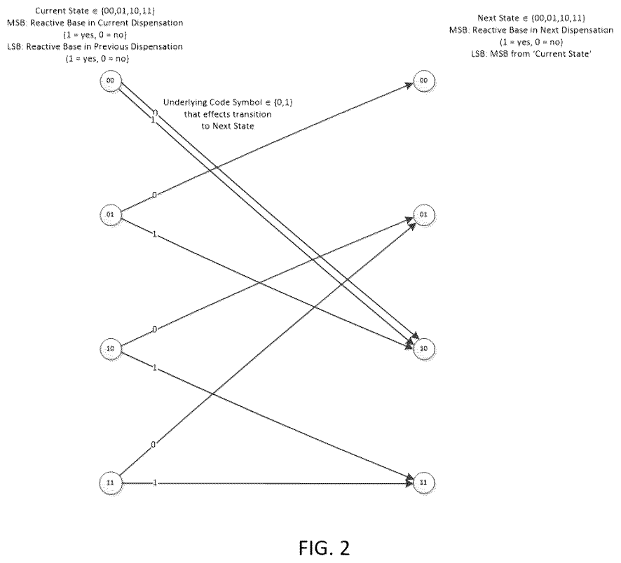

FIG. 2 illustrates an encoding trellis for a 4-bases-per-cycle pyrosequencing. The techniques may be used for encoding 3-cycle, 3-base-alphabet, and 5+-cycle, 5-and-higher-alphabet oligo-polymer hybrid schemes.

Note the use of 4 states in the trellis. Each state represents previous mappings of the last two positions:
(i) both unpopulated, (00);
(ii) both populated, (11);
(iii) newest-populated and older-unpopulated, (10);
(iv) newest-unpopulated and the older populated, (01).

Transitions to next states may indicate an update which either does not populate or does populate the next position in a sequence.

Four (4) states may be used to correctly implement a pyrosequencing scheme that is homopolymer-free; one position is populated every 3 positions. Note that if 3 consecutive positions were allowed to be unfilled, then the 4^{th} position may need to be filled (because an unhybridized duplex sequence may have an opening to at least one of the four nucleotides). That 4^{th} position being filled may result in generation of a homopolymer (repeat) of bases in a sequence—since the last filled base was the same base in the cycle before.

This aforementioned restriction may explain the double transition from the 00 state to the 10 state in the trellis diagram. A current state of 00 transitioning to a next state of 00 may imply 3 positions in a row were unfilled.

Optimal error correction codes may be constructed to maximize distance between their sets of codes. They are not constrained to disallow runs of three consecutive zeros. That may reduce the degrees of freedom they use to maximize distance. By contrast, the mappings to pyrosequenced positions comply with homopolymer-free and pyrosequencing constraints.

All other transitions in the picture design trellis may be natural results of populating a position with a ‘0’ or a ‘1’ and updating the next state to reflect that transition. Since 7 of the 8 transitions in the trellis perfectly express the underlying error correction code's structure, such a code can be quite effective and powerful.

Weakening transitions may occur when the underlying code has 3 consecutive zeros. One way to reduce those appearances is to use the sorting methodology described above. This method modestly reduces the library of codes. This method also ensures that the pyro-mapped codes that best reflect the underlying binary code's structure are faithfully reproduced, while those least reflective are not.

Another method to improve the weakening due to transitions involves breaking up strings of zeros by interleaving the code. Within a code, the (systematic) information section of bits—which precede the redundant section of parity bits—are the bits where the most consecutive zeros are usually seen. One way to eliminate those strings of zeros is to interleave the entire code design, so that the parity and information bits are intermingled. All codes may be intermingled by the same interleaving pattern. The interleaving technique does not help for the all-zeros code, which is generated by almost all linear codes. The all-zeros code can be excluded from the code set.

For the purposes of the specification and claims, the codes of the inventive concepts that are based on an encoding trellis can be referred to herein as “trellis codes”.

### Amplifying and Reading Codes

In an encoded assay, a target analyte is detected based on association of the target with a code in a recognition element, and detection of the code is used as a proxy for detection of the target analyte. A variety of techniques may be used to amplify, detect and decode the codes.

In one embodiment, recognition elements comprising codes are amplified using rolling circle amplification (RCA) to produce DNA nanoballs that include many duplicates of the code. An RCA reaction may include one or more rounds of amplification to produce the nanoball product. A nanoball may be from about 10,000 to about 1,000,000 or more nucleotides in length. In some embodiments, a nanoball may be more than or equal to 1,000, 5,000, 10,000, 15,000, 25,000, 50,000, 100,000, 200,000, 300,000, 400,000, 500,000, 600,000, 700,000, 800,000, 900,000, 1,000,000, 1,000,100, 1,000,200, 1,000,300, 1,000,400, or 1,000,500 nucleotides in length. In some embodiments, a nanoball may be less than or equal to U.S. Pat. Nos. 1,000,500, 1,000,400, 1,000,300, 1,000,200, 1,000,100, 1,000,000, 900,000, 800,000, 700,000, 600,000, 500,000, 400,000, 300,000, 200,000, 100,000, 50,000, 25,000, 15,000, 10,000, 5,000, or 1,000 nucleotides in length. A nanoball may include from about 100 to about 10,000 or more copies of the amplified code and other sequences of the amplified recognition element. In some embodiments, a nanoball may include more than or equal to 50, 100, 250, 500, 1,000, 2,500, 5,000, 7,500, 10,000, 12,500, or 15,000 copies of the amplified code and other sequences of the amplified recognition element. In some embodiments, a nanoball may include less than or equal to 15,000, 12,500, 10,000, 7,500, 5,000, 2,500, 1,000, 500, 250, 100, or 50 copies of the amplified code and other sequences of the amplified recognition element.

In one embodiment, the recognition elements comprising the codes may be amplified using a linear PCR amplification reaction to generate double stranded DNA amplicon products.

In one embodiment, recognition elements comprising codes may be amplified using bridge amplification to produce clusters of oligos on a surface.

In one embodiment, recognition elements comprising codes may be amplified on bead surfaces to produce bead-attached amplification products.

In one embodiment, the amplified codes of a recognition element may be determined based in part on a sequencing reaction.

In one embodiment, codes of a recognition element may be detected using a patterned array, such as a microarray comprising affixed oligonucleotides which are complimentary to all or a portion of the codes.

In one embodiment, codes of a recognition element may be detected in situ, e.g., in a cell or a tissue.

In one embodiment, in situ detection of a code of a recognition element may comprise determining the code based in part on a sequencing reaction.

In one embodiment, codes of a recognition element may be detected using an electronic/electrical sensing mechanism.

A variety of techniques and models may be used to detect and decode a nucleic acid code of a recognition element. In one embodiment, the inventive concepts provide models that make use of hard decision decoding methods or models. In another embodiment, the inventive concepts provide models that make use of soft decision decoding methods or models.

When using soft decision decoding techniques, it may not be necessary for the model to identify each nucleotide of the code specifically. For example, signals generated during each nucleotide addition cycle of a sequencing process may be detected and recorded to produce a data set that may be used as input into a model to calculate a probability that a specific code is present without requiring a hard decoding model. Although it may not necessary in a soft decision decoding model to make a hard decision about the identity of each nucleotide which results from cycle sequencing, a model developed according to the methods of the inventive concepts may nevertheless include a model for assigning a probability or identity to each nucleotide in the sequence of a code.

For soft decision decoding, it may not be necessary to identify each base specifically. For example, signals generated during each detection event may be detected and recorded to produce a data set that may be used as input into a model to calculate a probability that a specific code is present without requiring that each base of a code be determined. Although it may not be necessary in a soft decision decoding model to make a hard decision about the identity of each nucleotide, a model may nevertheless include assigning a probability or identity to each nucleotide in the sequence of a code, wherein each nucleotide in the sequence of a code may be sequenced. Data gathered includes intensity readings for signals produced by the hybridized detection polynucleotide fluorescent moiety in various spectral bands. A set of intensity readings are detected by imaging, stored and used as input into a soft decision decoding model for determining a probability that a particular code is present, and hence a target nucleic acid is present in the sample.

Data gathered during a sequencing process may, for example, include intensity readings for signals produced by the sequencing chemistry in various spectral bands. For example, in some cases the data is collected across a set of spectral bands that corresponds to part or all of the spectral bands expected to be produced by a series of nucleotide extension operations during a sequencing process.

In some embodiments, it may not be necessary to filter light from each nucleotide extension operation in order to distinguish between the nucleotides. Instead, a set of intensity readings may be detected, stored and used as input into a model for determining a probability that a particular code is present. In other embodiments, one or more filters may be used to refine signals from a sequencing process.

A model may be developed or trained using sequencing data from known codes, such as signal intensity data across a predetermined spectrum, during a sequencing process. The model may be used to calculate a set of probabilities across a set of one or more codes, indicating, for example, for each code, a probability that it is present in a sample.

In some cases, the model is developed or trained using data corresponding to color intensity signals across multiple color channels. In some cases, the model is developed or trained using data corresponding to color intensity signals across four color channels, each generally corresponding to the signal produced by addition of one of the four nucleotides A, T, C or G during a sequencing process. As discussed herein, the channels may experience color crosstalk.

A model may be built using data obtained using multiple light sensing channels. Each channel may be specific for a specific frequency bandwidth. In some cases, the model may be built using four channels, wherein the bandwidth of each channel may be selected for signals produced by addition of one of the four nucleotides A, T, C or G. In other cases, more or less than four channels may be used to collect data used to produce the model.

In certain embodiments of the inventive concepts, each channel detects a bandwidth region of a fluorescence signal produced by addition of one of four fluorescently labelled nucleotides. Nevertheless, the bandwidth of the signal produced by addition of one of four nucleotides may be spread across a spectral band that overlaps with other channels. In some embodiments, the emission spectrum is detected at varying intensities by multiple channels. In some embodiments, the emission spectrum is detected at varying intensities in some channels, but not others. Non-limiting examples of light sensing channels of the present disclosure are provided in U.S. application Ser. No. 18/391,323, which is hereby incorporated by reference.

As discussed in the examples herein, a color crosstalk model may be empirically developed and used as input into the model of the inventive concepts for producing a probability that a code is present. Relative coefficient strength may be experimentally determined across color channels for signal produced by addition of each nucleotide (A, T, C, G) from empirically produced test data.

Other factors that may be included in a statistical model according to the inventive concepts for calculating a probability that a code is present include signal phasing, signal droop, color cross-talk values, fluctuations in in color cross-talk values, noise, amplitude noise, gaussian amplitude models, and base calling algorithms.

The model of the inventive concepts may also account for various sources of noise and error, such as variability in the concentration of the active molecules in the assay, variability in color channel response due primarily to limited ability to estimate the color channel responses individually for each SBS cluster, and background and random error noise sources. A concentration noise model may be used to model the variable density of active molecules for a given cluster. A transduction noise model may be included to model variability in the color crosstalk matrix.

Accurately modeling the biochemical opto-mechanical processes in DNA sequencing is a complex process. Furthermore, to derive the inputs for a soft decision probabilistic signal estimator may include estimating the parameters driving the model, as well as having strong confidence that the model is accurate. Under these two assumptions, metrics can be computed that work directly with the received signals. In the commercially available base call algorithms, channel distortion effects are compensated for before the decision process; however, in soft decision decoding of the inventive concepts it is not necessary to compensate for distortions before decoding. Embodiments which do not compensate for distortions before decoding will have the advantage of avoiding information loss compensations, such as inversions.

The probability that a particular code is present is indicative of the probability that a particular target associated with a recognition element is present. Data indicating the probability that a particular target is present may be used, for example, to calculate probabilities relevant to diagnosis or screening of various medical conditions, or selection of drugs for treatment of various medical conditions.

The disclosure provides encoded probes, encoded recognition elements, that can be decoded using soft decision decoding methods or models. The codes may be generated using the trellis method and the codes may be referred to as “trellis codes”. The recognition elements of the inventive concepts may be padlock probes that include a soft decodable code, such as a trellis code. The recognition elements of the inventive concepts may be a dual probe that includes a soft decodable code, such as a trellis code.

The disclosure provides assays that make use of encoded probes or encoded recognition elements that may be decoded using soft decision decoding (“soft decoding”). In various embodiments, the assays make use of mixtures of recognition elements, each with a unique soft decodable code. A mixture may include 100s, 1,000s, 10,000s, 100,000s or more of encoded recognition elements.

In some instances of the methods of the inventive concepts, decoding a code is performed without making a specific base call for each nucleotide in the code.

In some embodiments, a hybridization-based detection method may be used to detect the code. In one embodiment, the amplified codes are identified using oligonucleotide probes in a hybridization-based reaction. The amplified codes may be identified using detection by hybridization. In one example, the hybridization-based detection method uses fluorescently labeled oligonucleotide probes. The code data may then be used as a digital count of the target-specific detection events.

### Assays

The encoded assays may make use of recognition elements or encoded probe sequences (“encoded probes”) for detecting one or more target analytes (“targets”). In some embodiments, the encoded probes are configured to interrogate two or more genomic ROIs of the target simultaneously. Such encoded probes are herein referred to as “multi-region encoded probes.”

An assay using multi-region encoded probes (e.g., an encoded assay) may include: (i) a recognition event, in which two or more genomic ROIs of a target are uniquely recognized and bound by a recognition element associated with a code (e.g., an encoded probe); (ii) a transformation event, in which a molecular transformation of the recognition element produces a modified recognition element comprising the code that may be used to provide a measure of the presence or absence of the target; and (iii) a detection event, that uses the code as a proxy for detection of the target, e.g., by recognizing, detecting and decoding the code (and optionally other elements).

An encoded assay may be a solution-based assay.

An encoded assay may be a surface-bound assay, e.g., on a substrate, a flow cell or on beads.

An encoded assay may be a hybrid assay that includes a surface-bound component and a solution-based component.

An encoded assay may be performed in a plate-based format (e.g., a multi-well plate, such as a 96-well plate). The multi-well plate may include, for example, a plurality of nanowells.

An encoded assay may be performed on a microfluidics device.

The encoded probe may include other functional sequences such as sequencing primer binding sites, one or more amplification primer binding sites, unique molecular identifier sequences (UMIs) and sample indexes. The sequencing primer binding sites may, in some cases, be adjacent to the code sequence. The amplification primer binding sites may, in some cases, be universal primer binding sequences that are common to all encoded probes in a set of encoded probes.

An encoded probe may be a recognition element, which may be a padlock probe. In some embodiments, the code may be a soft decodable code, such as a trellis code.

Thus, for example, the disclosure provides a recognition element in which the terminal sequences comprise target specific sequences and a soft decodable code is provided between the terminal sequences. Similarly, the disclosure provides a recognition element in which the terminal sequences comprise target specific sequences and a trellis code is provided between the terminal sequences. The disclosure provides a set of 10 or more recognition elements in each of which (A) the terminal sequences comprise target specific sequences and (B) a soft decodable code is provided between the terminal sequences. The disclosure provides a set of 100 or more recognition elements in each of which (A) the terminal sequences comprise target specific sequences and (B) a soft decodable code is provided between the terminal sequences. The disclosure provides a set of 1000 or more recognition elements in each of which (A) the terminal sequences comprise target specific sequences and (B) a soft decodable code is provided between the terminal sequences. The disclosure provides a set of 10,000 or more recognition elements in each of which (A) the terminal sequences comprise target specific sequences and (B) a soft decodable code is provided between the terminal sequences. In certain embodiments, the foregoing sets are provided in the absence of any recognition elements that do not include the soft decodable codes. In certain embodiments, the foregoing sets are provided with codes that are homopolymer-free and soft decodable.

In some embodiments, an encoded probe may be a molecular inversion probe. The code may be a soft decodable code, for example, the code may be a trellis code.

The disclosure provides a set of 10 or more molecular inversion probes in each of which (A) the terminal sequences comprise target specific sequences and (B) a soft decodable code is provided between the terminal sequences. The disclosure provides a set of 100 or more molecular inversion probes in each of which (A) the terminal sequences comprise target specific sequences and (B) a soft decodable code is provided between the terminal sequences. The disclosure provides a set of 1000 or more molecular inversion probes in each of which (A) the terminal sequences comprise target specific sequences and (B) a soft decodable code is provided between the terminal sequences. The disclosure provides a set of 10,000 or more molecular inversion probes in each of which (A) the terminal sequences comprise target specific sequences and (B) a soft decodable code is provided between the terminal sequences. In certain embodiments, the foregoing sets are provided in the absence of any molecular inversion probes that do not include the soft decodable codes. In certain embodiments, the foregoing sets are provided with codes that are homopolymer-free and soft decodable.

The transformation event may include a ligation or gap-fill ligation reaction to produce the modified recognition element comprising the code.

The detection event may include an amplification operation in which the code sequence (among other elements) is amplified in an amplified recognition element. Amplification may be by any method of amplification, including for example, on-surface PCR, isothermal amplification, rolling circle amplification (RCA), multiple strand displacement amplification, ultrarapid amplification, or any combination thereof. Surface based amplification may be performed using PCR with surface-anchored primers (e.g., Illumina® bridge amplification technology) or recombinase polymerase amplification (RPA) (e.g., ExAmp technology), or the like.

In one embodiment, the amplification operation comprises a rolling circle amplification (RCA) reaction to generate a nanoball product. In one embodiment, the amplification operation comprises rolling circle amplification (RCA) on an anionic surface to generate a nanoball product. In one embodiment, the amplification operation comprises rolling circle amplification (RCA) on a polylysine surface to generate a nanoball product. In one embodiment, the amplification operation comprises rolling circle amplification (RCA) on an anionic surface without covalently attaching the modified recognition element to the surface to generate a nanoball product. In one embodiment, the amplification operation comprises rolling circle amplification (RCA) on a polylysine surface without covalently attaching the modified recognition element to the surface to generate a nanoball product.

In one embodiment, an encoded probe may include a sequence which may prevent RCA of the encoded probe, thereby allowing for production of linear double-stranded PCR products. The non-extendable sequence may, for example, be located between a pair of amplification primer binding sequences.

In one embodiment, an encoded probe may include a restriction enzyme site that may be cleaved to yield a linear DNA molecule.

### Other Amplification Strategies

In some embodiments, the amplified recognition element comprising the code may be sequenced to identify the sequence of the code associated with the recognition element and hence the target. Any sequencing technology may be used to sequence the RCA products. Non-limiting examples of sequencing technologies that may be used include sequencing by synthesis (e.g., pyrosequencing; sequencing by reversible terminator chemistry (Illumina®)), avidity sequencing (Element Biosciences), sequencing by hybridization, sequencing by ligation, and nanopore sequencing.

In some embodiments, a sequencing library may be generated from a set of modified recognition elements comprising the codes. The library may be sequenced to determine the code associated with the recognition element and hence a target of interest. The code data may then be used as a digital count of the target-specific detection events. In some embodiments the code is a soft-decodable code.

In one embodiment, a sequencing library comprising the code (among other elements) may be generated from a circularized recognition element.

In one embodiment, a sequence library comprising the code (among other elements) may be generated from a nanoball product generated by performing RCA on a circularized recognition element.

In one embodiment, a nanoball or a portion of the nanoball that includes the code (and other elements) may be directly sequenced to determine the code associated with the recognition element and therefore the target of interest. The code data may be used as a digital count of the target-specific detection events.

In some embodiments, a hybridization-based detection method may be used to detect the code. In one embodiment, the amplified codes are detected using oligonucleotide probes in a hybridization-based reaction such as, for example, detection by hybridization. In one example, the hybridization-based detection method uses fluorescently labeled oligonucleotide probes. The code data may then be used as a digital count of the target-specific detection events. Decoding of the fluorescence data generated by utilizing a detection by hybridization approach may be soft decision decoding.

### Coded Multi-Region Recognition Elements

The disclosure provides assays that make use of coded multi-region recognition elements comprising codes that may be used as a proxy for detection of two or more genomic ROIs in a target, e.g., by recognizing and decoding the associated code. The code in a multi-region recognition element may be a soft decodable code (e.g., a trellis code). A coded multi-region recognition element may include target-specific regions that may be used for target recognition and enrichment. The coded multi-region recognition element may include regions configured to hybridize to two or more genomic regions of interest (ROI) in a target. A coded multi-region recognition element may include a 5′ terminal phosphate that may be used to facilitate ligation (e.g., circularization) after target hybridization. A coded multi-region recognition element may include a 3′ nucleotide that is the complement to a nucleotide at a target site of interest (e.g., a 3′ SNP-specific nucleotide). A coded multi-region recognition element may include an RCA primer binding site that includes a primer binding sequence suitable for priming an RCA reaction.

For example, the coded multi-region recognition element may include regions at the 3′ and 5′ ends that are complementary to regions of a target. The 3′ and 5′ end regions may hybridize to the target, and the probe may be circularized, e.g., by a ligation or gap-fill ligation reaction. As described here, the target may be a nucleic acid analyte (e.g., mRNA, DNA etc.) or a proxy for the analyte of interest (e.g., an oligonucleotide conjugated to an antibody).

Non-limiting examples of coded recognition elements of the present disclosure are provided in U.S. application Ser. No. 18/391,323, which is hereby incorporated by reference. The present disclosure provides coded recognition elements configured to detect two or more genomic ROIs in a single target (“multi-region recognition element”). In some embodiments, the coded multi-region recognition element may include a 5′ target specific region and a 3′ target specific region that are complementary to regions of a target. For example, the coded recognition element may be an oligonucleotide having a 5′ probe arm and a 3′ probe arm, wherein each of 5′ probe arm and 3′ probe arm comprise binding sites for two or more genomic ROIs. The target may be a nucleic acid analyte (e.g., mRNA, DNA, etc.) or a proxy for the analyte of interest (e.g., an oligonucleotide conjugated to an antibody). The target may have two or more genomic ROIs, such as a variation in a nucleotide sequence. The variation in a nucleotide sequence may be, for example, a polymorphism. Non-limiting examples of polymorphisms include single nucleotide polymorphism (SNP), single nucleotide variants (SNV), indels (insertions/deletions), and copy number variants (CNV). The genomic ROI may not be a variation in the target. For example, the coded multi-region recognition element may bind to a nucleotide or nucleic acid sequence that is a wild-type sequence. The genomic ROI may be the major allele or a minor allele in a given population. The two or more genomic ROIs may comprise both a wild-type and a variant nucleotide or nucleic acid sequence in the target. For example, 5′ probe arm of the coded multi-region recognition element may include a binding site for a wild-type nucleotide and a 3′ probe arm may include a binding site for a variant nucleotide. Alternatively, the opposite may be true and 3′ probe arm of the coded multi-region recognition element may include a binding site for a wild-type nucleotide and a 5′ probe arm may include a binding site for a variant nucleotide.

The coded multi-region recognition element of the present disclosure may utilize a bridge element. In some embodiments, the bridge element is a synthetic oligonucleotide that is complementary to a target sequence. In some embodiments, the bridge element is complementary to a region of the target sequence interposed between the region of the target complementary to, or hybridized to, 5′ target specific region and 3′ target specific region of the coded multi-regional recognition element. In some embodiments, the bridge element may be DNA or cDNA.

The 5′ target specific region may include a 5′ terminal phosphate (P) that may be used to facilitate ligation (e.g., circularization) after target recognition and hybridization. The 3′ target specific region may include one or more terminal 3′ nucleotides “N” complementary to a nucleotide at a target site of interest. The 5′ target specific region may include one or more nucleotides “N” complementary to another nucleotide at a target site of interest. For example, the nucleotide “N” in 5′ target specific region and the nucleotide “N” in 3′ specific region may be for two SNP specific nucleotides present in the same locus of a target.

The 5′ and 3′ target specific regions may hybridize to the target, and the probe may be circularized. For example, when the complementary nucleotide is present in the target, 3′ SNP specific nucleotide hybridizes to the target, enabling circularization, e.g., by ligation or gap-fill ligation. Other types of features or mutations may be detected by varying the terminal nucleotide (N) or nucleotides of a target specific region and/or target specific regions to hybridize when the target of interest is present and not hybridize when the target of interest is not present.

The coded multi-region recognition element may include an RCA priming binding site that includes a primer sequence suitable for priming an RCA reaction. For example, the RCA priming binding site may be downstream from a target specific region. However, other locations are possible, as long as the positioning of the primer binding site does not interfere with the other functions of the recognition element, e.g., the recognition element hybridization function and the encoding function.

A coded multi-region recognition element may optionally include other functional sequences. For example, the recognition element may include index sequences which are unique identifiers present in the recognition element sequence or inserted as part of the assay. Index sequences, such as sample barcodes, can allow for differentiation among different samples, experiments, etc. during a detection event.

The coded multi-region recognition element may include unique molecular identifiers (UMIs). UMIs may be inserted anywhere within the recognition element to address downstream readout and data analysis purposes. For example, UMIs may be introduced to distinguish unique recognition events with single-molecule resolution during the detection operation. UMI's may facilitate error correction and/or individual molecule counting.

A coded multi-region recognition element may include other primer binding sites in addition to the priming region for RCA amplification. Other priming regions may, for example, be present to facilitate the detection of an index, a UMI or other sequences present in the recognition element. Priming regions may allow parallel or serial decoding schemes. They may also be used to increase the amount of multiplexing or allow sequential decoding. For instance, if a plurality of probes or amplified objects are present, those containing a specific primer may be amplified or read. Primers may also be used to facilitate the capture and immobilization of a probe or amplified object onto a surface (e.g., via DNA-DNA hybridization).

A coded multi-region recognition element may include one or more sequences recognizable by enzymes, such as endonucleases. Various sequences may be selected and used to facilitate additional transformations, such as digestion, nick or gap formation, phosphorylation etc. In one embodiment, the recognition element includes one or more restriction sites.

A coded multi-region recognition element may include one or more non-natural nucleotides. Non-limiting examples include phosphorothioate groups, locked DNA (LNA), peptide DNA (PNA) and others, which may be included to improve certain features of the recognition element, such as melting temperature for target recognition, or primer recognition, or resistance to degradation. Additionally, abasic nucleotides (“wobble bases”) may be included in the recognition element sequence to add degeneracy to targeting or priming regions and extend the ability to recognize a broader number of complementary sequences.

A coded multi-region recognition element may include one or more chemical moieties. Such chemical moieties may be included in the recognition element structure or added at any stage of the workflow to enable additional transformations or properties. Non-limiting examples include cleavable groups to open or linearize the recognition element, reactive groups to add additional components such as dyes, and groups to facilitate immobilization on surfaces.

A coded multi-region recognition element may include CRISPR recognition sequences, sequences designed to be recognized by CRISPR enzymes and replaced with other arbitrary sequences. The recognition element may optionally include one or more sequences designed to be recognized by transposases and replaced with other arbitrary sequences.

A coded multi-region recognition element may optionally include one or more adapter primers for compatibility with sequencing by synthesis (SBS) and other non-SBS platforms. The adapter primers may be included in the recognition element sequence or added at any stage as part of the workflow. Such adapter primers may be used directly to immobilize, cluster, extend, and amplify as precursor activities to a sequencing run by SBS or another non-SBS method.

In one embodiment, a recognition element assay workflow may include:
(i) hybridizing the recognition element to a target;
(ii) optionally, extending the hybridized recognition element to fill any single-stranded gap ( ) remaining between the two recognition element arms;
(iii) circularizing the recognition element when the target analyte is present;
(iv) removing (e.g., by exonuclease or other mean) non-circularized recognition elements remaining after ligation;
(v) amplifying the circularized recognition element by RCA or other methods;
(vi) capturing of the amplified product on a surface;
(vii) degrading the amplified product to generate a sequencing compatible library;
(viii) preparing the library for sequencing, using sequencing sample preparation workflows suitable for a desired sequencing platform; and reading out or decoding the code.

### Index Sequences

Index sequences, such as sample barcodes, allow differentiation among different samples, experiments, etc. during a detection event (e.g., reading or decoding the code). Indexes may be added to a recognition element using a variety of strategies.

Indexes may be added during the synthesis of a recognition element (e.g., padlock probe). In this case, for every padlock probe manufactured, the number of padlock probes is N×P, where N is the number of indices and P is the plexity of the padlock probe pool.

Indexes may be added after recognition element synthesis as part of manufacturing or at a site of use as an operation prior to performing an encoded assay. In this case, one synthesis may be included for each padlock probe and additional functional elements. Additional functional elements may be added to a padlock probe to enable insertion of an index. Examples of functional elements that may be added include (i) non-natural nucleotides (e.g., biotin, amine, etc.) and (ii) polynucleotides that enable biochemical transformation of the padlock probe to include an index sequence such as adapters for ligations or extension ligations, restriction endonuclease recognition sites, and transposome binding sites.

Indexes may be added during an encoded assay. For example, a ligation reaction to insert an index can occur at the same time as ligation of the padlock probe at the target site of interest to generate a circularized padlock probe (e.g., the transformation event). In some cases, the ligation reaction may be a gap-fill extension/ligation reaction.

Indexes may be added after ligation of the padlock probe and RCA by including modified nucleotides during the RCA reaction. The modified nucleotides may be coupled to an index sequence. In cases where there is a covalent or non-covalent interaction, either moiety can be linked to the index sequence or incorporated during RCA.

Non-limiting examples of coupling strategies include: (i) ligand protein pairs such as biotin-streptavidin, antigen-antibody, CLIP tag and SNAP tag pair (e.g., 06-benzylguanine derivatives coupling to 06-alkylguanine-DNA-alkyltransferase, wherein either the protein or the substrate may be bound to the probe), carbohydrate-protein pairs (e.g., lectins), and digoxigenin-DIG-binding protein; (ii) peptide-protein pairs (e.g., SpyTag-SpyCatcher); and (iii) hybridizing indexes to a common sequence on the RCA product.

Indexes may be added to RCA products by restriction endonuclease cleavage followed by index ligation.

Indexes may be added to RCA products using a transposase enzyme that fragments and indexes the RCA products.

Non-limiting examples of index sequences of the present disclosure are provided in U.S. application Ser. No. 18/391,323, which is hereby incorporated by reference.

In some embodiments, the index sequence attached to a recognition element (e.g., padlock probe) may be performed using a ligand protein coupling strategy. In this example, the ligand protein pair may be biotin-streptavidin. Biotinylated nucleotides “B” may be incorporated into a padlock probe and an index sequence may be attached to a streptavidin protein. Index sequence may then be coupled to the recognition element via formation of a streptavidin—biotin linkage.

In another example, an index sequence may be added to a padlock probe by restriction endonuclease cleavage followed by index ligation. A recognition element may include a pair of restriction sites. A polymerase extension reaction may be performed to convert a padlock probe to a double-stranded molecule prior to cleavage. An index sequence may be added to a padlock probe by restriction endonuclease cleavage followed by index ligation.

### Surface Attachment

The encoded assays of the inventive concepts herein may be performed on a surface. For example, a target may be immobilized on a surface for conducting assays of the inventive concepts. The recognition elements of the inventive concepts may be immobilized on a surface for conducting assays of the inventive concepts. DNA nanoballs of the inventive concepts may be immobilized on a surface for conducting assays of the inventive concepts. Various intermediate assemblies of molecules of the assays of the inventive concepts may be immobilized on a surface for conducting assays of the inventive concepts.

Various operations of the inventive concepts may be performed on a surface, such as target capture, recognition events, transformation events, amplification, and/or detection events, e.g., determination of the absence or presence of the code (e.g., by sequencing or hybridization-based detection).

Thus, for example, the disclosure provides a surface having a recognition element as described herein immobilized on the surface. The disclosure provides a surface having a nanoball as described herein immobilized on the surface. The disclosure provides a surface having a target of interest immobilized on the surface. The disclosure provides a surface having a target immobilized on the surface with a recognition element as described herein hybridized to the target. The disclosure provides a surface having a recognition element immobilized on the surface with a target as described herein hybridized to the recognition element. The disclosure provides a surface having a target nucleic acid immobilized on the surface, and a protein or peptide bound to the target nucleic acid. The disclosure provides a surface having a target nucleic acid immobilized on the surface, and an antibody, aptamer, binder, or antibody fragment bound to the target nucleic acid. The disclosure provides a surface having a ligand that has affinity for any of the foregoing immobilized on the surface. For example, the ligand may have affinity for a recognition element as described herein, a nanoball as described herein, or a target as described herein. The ligand may, for example, be a protein, peptide, antibody, aptamer, binder, or antibody fragment.

A variety of surfaces may be used for the surface attachments described herein. In various embodiments, the surface includes, but is not limited to, an oxide, a nitride, a metal, an organic or an inorganic polymer (e.g., hydrogel, resin, plastic or other).

The surface may take a variety of forms, e.g., the surface may be flat or curved. The surface may include beads or particles. In some cases, the surface may be the surface of a flow cell. Beads or other particles may, in some embodiments, range in size from less than 100 nanometers (nm) up to several centimeters.

Various surface modifications may be used to permit attachment of various components of the assays of the inventive concepts to a surface. For example, various anchoring ligands may be used (e.g., streptavidin, biotin, aptamers, antibodies, etc.). Chemical handles, such as click chemistry handles, may be used. Non-limiting examples include azides, alkynes, unsaturated bonds, amines, carboxylic acids, NHS, DBCO, BCN, tetrazine, epoxy and the like. Single- or double-stranded oligonucleotides may be used. Size ranges of the oligonucleotides may, in some cases, be from about 10 to about 200 nucleotides. Proteins or peptides may be used for surface attachment. Charge-based molecules or polymers may be used, e.g., polyethylenimine. In some embodiments, the surface may be modified with polylysine.

Various techniques may be used to prepare a surface for binding to a target or to a component of an assay of the inventive concepts. In one example, a flow cell with primers may be used. A splint DNA segment that comprises a segment complementary to the primer and a segment that is complementary to the target, or the component of the assay may be hybridized to the primer. A variety of splints may be used on a surface, with various subsets of the splints having different segments complementary to different components of the inventive concepts or different targets. Specific splints may be arranged on different regions of a surface. For example, splints may be arranged in a manner that permits the identification of distinct regions of a surface targeted to specific analytes or components of the assays.

In various embodiments, amplification of a nucleic acid may occur on the surface. The nucleic acid may be a target or any nucleic acid component of an assay of the inventive concepts. For example, a target analyte may be amplified on a surface, or a recognition element (modified or otherwise) of the inventive concepts may be amplified on a surface, and/or a fragment of any of the foregoing may be amplified on a surface. The amplification may be performed on a bead or particle, or on a flat surface, such as on the surface of a flow cell.

It should also be noted that DNA may be amplified in solution, e.g., in an aqueous suspension or emulsion, such as in microdroplets. Solution-based amplification may be performed, for example, in an open environment, such as the well of the microtiter plate, in a nanowell, or in an enclosed space, droplet in an emulsion, or on a flow cell or other microfluidic device.

Amplification may be by any method of amplification, including for example, PCR, isothermal amplification, multiple strand displacement amplification, rolling circle amplification (RCA), ultrarapid amplification, or any combination thereof.

Attachment for immobilization of components of the assays or of targets may be covalent or non-covalent (e.g., Coulombic in nature), temporary or permanent, and/or rendered labile when subject to a particular stimulus.

Non-limiting examples of mechanisms of lability include:
Enzymatic—protease, restriction endonuclease, CRISPR-Cas9
Chemical—reduction, hydrolysis, nucleophilic attack, displacement, reducing of a disulfide bond
Temperature—melting of duplexed hybridized DNA, thermodynamically unfavorable conditions (Positive deltaG)
pH—hydrazone, carbonate, etc.
Light—O-nitrobenzyl or derivatives where absorption of light of a particular wavelength(s) can cause bond rearrangements or cleavage. Light sensitive groups include nitro-benzene derivatives
Ligand mediated—competitive competition for binding site (see examples below)·
   Peptide-tagged oligos with protein interactions—e.g., Spy-catcher. The moiety may be the ligand or the protein.
   Peptide-tagged oligo with heavy metal interactions—e.g., Hexa-histidine—to Cu. The moiety may be the ligand or the protein.
   CLIP tag and SNAP tag pair—e.g., O6-benzylguanine derivatives coupling to O6-alkylguanine-DNA-alkyltransferase. Either the protein or the substrate may be bound to the oligo.
   Carbohydrate-protein pairs, e.g., lectins
   The moiety may be a ligand (e.g., biotin, digoxigenin) coupled to a fluorescently-tagged protein (e.g., avidin, streptavidin, DIG-binding protein)
Cleavage can be performed by cleaving a moiety dangling on a nucleotide, or a nucleotide or a nucleobase within the oligo sequence or the di-nucleotide linkage, e.g., uracil and USER cocktail (uracil-N-deglycosylase (UNG)) followed by Endonuclease VIII or FPG (Formamidopyrimidine DNA Glycosylase with Bifunctional DNA glycosylase with DNA N-glycosylase and AP lyase activities)
Cleavage can be performed by an enzyme

### Surface-Based Workflows

A variety of surface-based workflows may be used within the scope of the assays disclosed herein. In some embodiments, a surface-based workflow may use a recognition element (e.g., padlock probe) configuration that includes a recognition element associated with a code. The code may be a soft decodable code, such as a trellis code. In some embodiments, a surface-based workflow may use a dual probe recognition element that includes a recognition element associated with a code (e.g., a trellis code).

In some embodiments, a surface-based workflow may include immobilizing a target on a surface and hybridizing a recognition element to the target. In one embodiment, a surface-based workflow may include:
(i) immobilizing the target on a surface;
(ii) hybridizing a recognition element to the immobilized target;
(iii) circularizing the recognition element to produce a circular modified recognition element; and
(iv) releasing the circular modified recognition element from the target.

In some embodiments, the target may be a nucleic acid, e.g., DNA. In this case, immobilization of the nucleic acid target (e.g., DNA) may be at an end of the target or via a side chain or internal segment of the target.

Non-limiting examples of surface-based workflows of the present disclosure are provided in U.S. application Ser. No. 18/391,323, which is hereby incorporated by reference. In some embodiments, the workflows comprise immobilizing a target to a surface. For example, a target may be immobilized on a surface by an anchor element. In one example, target is DNA and anchor element is an oligonucleotide. In some embodiments, the workflows comprise hybridizing the linear recognition element to the immobilized target. For example, a solution that includes a recognition element may be added, and a hybridization reaction may be performed to hybridize the recognition element to the target. In one example, the recognition element is a coded multi-region recognition element. In some embodiments, the workflows comprise circularizing the coded multi-region recognition element. For example, a ligation reaction may be performed to circularize the coded multi-region recognition element to produce a circular modified coded multi-region recognition element. In some cases, a gap-fill extension/ligation reaction is used to circularize the coded multi-region recognition element to produce the circular modified coded multi-region recognition element. In some embodiments, the workflows comprise releasing the circular modified coded multi-region recognition element from the immobilized target for downstream processing. For example, a circular modified coded multi-region recognition element may be dehybridized from the target and amplified in an RCA reaction to produce a nanoball product.

In some cases, the RCA reaction may be performed in a solution that remains in contact with the surface on which the target is immobilized (e.g., in the same container, well, reservoir, liquid volume or droplet). In some cases, the solution comprising the released modified coded multi-region recognition element may be transferred to a separate container prior to performing the RCA reaction. In some cases, the solution comprising the released modified coded multi-region recognition element may be transferred to a different surface prior to performing the RCA reaction.

In some embodiments, the immobilized target (e.g., DNA) may be used to prime the RCA reaction. In one embodiment, a surface-based workflow may include:
(i) immobilizing the target on a surface;
(ii) hybridizing a recognition element to the target;
(iii) circularizing the recognition element to produce a circular modified recognition element; and
(iv) using the target to prime an RCA reaction to generate a nanoball product.

In another example of a surface-based workflow, the target may be immobilized on the surface and used as a primer to initiate the amplification of the recognition element to generate a nanoball product. In some embodiments, the workflows comprise immobilizing the target analyte to the surface. For example, the target may be immobilized on the surface by an anchor element. In one example, the target is DNA and the anchor element is an oligonucleotide. In some embodiments, the workflows comprise hybridizing a linear recognition element to the immobilized target. For example, a solution that includes a recognition element (e.g., a multi-region coded multi-region recognition element) may be added and a hybridization reaction is performed to hybridize the recognition element to the target. In some embodiments, the workflows comprise circularizing the recognition element. For example, a ligation reaction may be performed to circularize the recognition element to produce a circular modified recognition element. In some cases, a gap-fill extension/ligation reaction is used to circularize the recognition element to produce the circular modified recognition element. In some embodiments, the workflows comprise using the immobilized target as a primer to initiate an RCA reaction to generate a nanoball product.

In some embodiments, a surface-based workflow may include immobilizing a recognition element (or a part thereof) on a surface and using the immobilized recognition element to capture a target. In one embodiment, a surface-based workflow may include:
(i) immobilizing the recognition element (or a part thereof) on a surface;
(ii) hybridizing a target to the recognition element;
(iii) circularizing the recognition element to produce a circular modified recognition element; and
(iv) using the target to prime an RCA reaction to generate a nanoball product.

In another example of a surface-based workflow, the recognition element is immobilized on the surface and the immobilized recognition element is used to capture a target. In some embodiments, the workflows comprise immobilizing a linear recognition element to a surface. For example, a recognition element may be immobilized on a surface by an anchor element. In one example, the recognition element is a multi-region recognition element and the anchor element is an oligonucleotide. In some embodiments, the workflows comprise hybridizing the target to the immobilized recognition element. For example, a solution that may include a target may be added and a hybridization reaction may be performed to hybridize the target to the recognition element. In some embodiments, the workflows comprise circularizing the recognition element. For example, a ligation reaction may be performed to circularize the recognition element to produce a circular modified recognition element. In some cases, a gap-fill extension/ligation reaction may be used to circularize the recognition element to produce the circular modified recognition element. In some embodiments, the workflows comprise amplifying the circular modified recognition element in an RCA reaction to generate a nanoball product. The circular modified recognition element may be amplified without being released from the surface. For example, a circular modified recognition element may be amplified in an RCA reaction using the target as a primer to initiate the amplification reaction.

In some embodiments, the circular modified recognition element may be released from the surface prior to amplification. In some cases, the RCA reaction may be performed in a solution that remains in contact with the surface on which the recognition element was anchored (e.g., in the same container, well, reservoir, liquid volume or droplet). In some cases, the solution comprising the released modified recognition element may be transferred to a separate container prior to performing the RCA reaction.

In some embodiments, the solution comprising the released modified recognition element may be transferred to a different surface prior to performing the RCA reaction. In one embodiment, oligonucleotides bound to the new surface may be used as capture moieties to immobilize the circular modified recognition element on the surface and to initiate the amplification reaction. In one embodiment, the target may be immobilized on the new surface and used to initiate the amplification reaction.

A surface-based workflow may use a dual probe as a recognition element. In one embodiment, a surface-based workflow using a dual probe may include:
(i) hybridizing a target to a first probe of the dual probe recognition element;
(ii) hybridizing the target to a second probe of the dual probe recognition element; and
(iii) performing a ligation or a gap-fill ligation reaction to link the first probe and the second probe.

In some embodiments, the first probe and the second probe may both be immobilized on the surface.

In some embodiments, the first probe is immobilized on the surface and the second probe is in solution. The surface may, for example, be the surface of a flow cell.

A non-limiting example of a multi-region dual probe recognition element workflow immobilizes a first probe of the dual probe recognition element on the surface and the second probe of the dual probe recognition element is in solution, wherein the first probe is configured to interrogate a variant in the target, and the second probe is configured to interrogate another variant in the target. In some embodiments, the workflows comprise hybridizing a target to the first probe immobilized on the surface. For example, a first probe may be immobilized on a surface via an anchor element. In one example, the anchor element is a surface bound primer. The surface bound primer may, for example, be a primer on a sequencing flow cell. A process for anchoring a first probe (or a segment thereof) on a surface bound primer is described herein. In some embodiments, the workflows comprise using the first probe as a capture element for recognizing and binding a target. For example, a solution that may include a DNA target may be added and a hybridization reaction is performed to hybridize the DNA target to the first probe. In some embodiments, the workflows comprise hybridizing the target to a second probe. For example, a solution that may include a second probe comprising a sequence for recognizing and hybridizing a DNA target is added and a hybridization reaction is performed to hybridize the second probe to the target. In some embodiments, the workflows comprise ligating the dual probe to link the first probe and the second probe to produce a modified dual probe recognition element immobilized on the surface. For example, a ligation reaction is performed to link the first probe and the second probe to produce a modified dual probe recognition element. In some cases, a gap-fill extension/ligation reaction is used to link the first probe and the second probe to produce the modified dual probe recognition element. In some cases, second probe may further include a surface oligonucleotide adapter for binding to another surface bound primer. In this example, a dual probe recognition element may further include a surface adapter that is introduced during ligation of the dual probe recognition element and to produce a modified dual probe recognition element.

The disclosure provides a process for preparing a surface for binding to a target or to a component of an assay of the inventive concepts. Surface modifications may serve a dual purpose. For example, a surface modification may (i) capture the target of interest and (ii) initiate the amplification of a recognition element or a portion thereof on the surface. In another example, a surface modification may (i) capture a component of the assay (e.g., a circular modified recognition element), and (ii) initiate an RCA reaction to generate a nanoball product.

A surface bound primer may be enzymatically modified to include a capture sequence. A capture sequence may be a target-specific probe or a sequence that is specific for a component of an assay. A surface bound primer may be enzymatically modified to include a recognition element or a portion thereof (e.g., a probe arm or a primer binding site). For example, a splint oligonucleotide that includes a segment that is complementary to a surface bound primer and a segment that is complementary to a recognition element (or a portion thereof) may be hybridized to the primer and used to template the synthesis of a surface bound recognition element. In one example, the surface bound probe is one arm of a dual probe recognition element.

Non-limiting examples of synthesizing a surface bound recognition element using a splint oligonucleotide of the present disclosure are provided in U.S. application Ser. No. 18/391,323, which is hereby incorporated by reference. In some embodiments, a surface is provided with a surface bound primer. For example, a primer is bound to a surface. The surface may, for example, be the surface of a flow cell. In some embodiments, a splint oligonucleotide is hybridized to the surface bound primer. For example, a splint that includes a segment that is complementary to a primer and a capture segment is hybridized to the primer. In one example, the capture segment is one arm of a multi-region dual capture recognition element. In some embodiments, a primer extension reaction is performed to synthesize the surface bound recognition element. For example, in the primer extension reaction, a splint is used to template the synthesis of a capture segment extending from the primer to produce a surface bound recognition element arm.

### Amplification Strategies

Amplification may be by any method of amplification, including for example, on-surface PCR, isothermal amplification, rolling circle amplification, multiple strand displacement amplification and/or ultrarapid amplification.

Surface based amplification may be performed using PCR with surface-anchored primers (e.g., Illumina® bridge amplification technology) or recombinase polymerase amplification (RPA) (e.g., ExAmp technology).

Clonally amplified material may be a nanoball or a DNA cluster (e.g., Illumina® surface-based amplification).

An amplification strategy may include adding a second surface adapter to a recognition element. The second surface adapter may be complementary to a second primer on a flow cell surface (e.g., a bridge amplification primer). The second surface adapter may, for example, be added to a recognition element during the ligation or gap-fill ligation event or added separately by PCR or through its own ligation to a recognition element. For example, an amplification strategy may include using the splint ligation approach to add a second surface adapter to a surface bound recognition element to facilitate bridge amplification. Bridge amplification may be used to create clusters of amplicons for sequencing, as described in U.S. application Ser. No. 18/391,323, which is hereby incorporated by reference.

An amplification strategy may include adding a restriction enzyme site to a recognition element. For example, the recognition element may include a restriction enzyme site that when hybridized with a complementary oligonucleotide provides a double-stranded site for a restriction endonuclease to cleave the recognition element, rendering a linear recognition element. The linear recognition element may be amplified for downstream processing, e.g., for sequencing. For example, the linear recognition element may be captured on a flow cell and amplified by bridge amplification (e.g., Illumina® bridge amplification technology) or recombinase polymerase amplification (RPA) (e.g., ExAmp technology).

The recognition element may include surface primer binding site sequences or surface adapter binding sequences that are complementary to surface bound primers of a flow cell. The adapter sequences may be linked to or adjacent to the restriction site, so that when the site is cleaved by a restriction enzyme the linear recognition element is ready for sequencing. As noted, other forms of cleavage are possible, such as CRISPR mediated cleavage or any other double-stranded break inducing protein.

For example, a recognition element that includes a restriction enzyme site may be used to linearize the recognition element for capture on a flow cell for bridge amplification prior to sequencing. In some embodiments, a recognition element may include a restriction site. Restriction site may be linked to a first surface adapter and a second surface adapter. An oligonucleotide that is complementary to a restriction site may be hybridized to a recognition element to provide a double-stranded site for restriction endonuclease cleavage. Cleavage at restriction site generates a linear recognition element. A linear recognition element may be loaded on a surface (e.g., a flow cell surface) that includes a first primer and a second primer immobilized thereon. Hybridization of an adapter to a primer may be used to initiate a bridge amplification reaction to generate clusters of amplicons for sequencing.

Similarly, a nanoball may include surface primers or sequencing adapters linked to or adjacent to a restriction site, so that when the site is cleaved by a restriction enzyme the linear strands are released ready for sequencing. As noted, other forms of cleavage are possible, such as CRISPR mediated cleavage.

In another embodiment, a nanoball with adapter sequences complementary to surface bound primers may be seeded directly onto the surface without cleaving. Amplification may proceed through bridge amplification (e.g., Illumina® bridge amplification technology) or recombinase polymerase amplification (RPA) (e.g., ExAmp technology) initiated directly.

Rolling circle amplification (RCA) may be used to produce nanoballs as part of the assays of the inventive concepts. An RCA reaction may be performed as a surface-bound reaction. For example, RCA may be initiated by an oligonucleotide bound to a surface (e.g., beads, flow cells, microwell, or nanowells). Any method may be used to bind the oligonucleotide to the surface. In one example, the oligonucleotide may be covalently bound to the surface. An oligonucleotide may be covalently attached to a surface. An oligonucleotide may include an RCA primer sequence that is complementary to an RCA primer binding site on a recognition element. An oligonucleotide may be used to capture a recognition element by hybridization of the complementary sequences and initiate the RCA reaction. Because the oligonucleotide is covalently bound to the surface, the surface-bound RCA reaction generates a nanoball that is covalently attached to the surface.

In another example, a cation-coated surface (e.g., beads, flow cells, microwells, or nanowells) may be used to capture nanoballs. In one example, the cation-coated surface may be a polylysine-coated surface. A surface may be coated with a polylysine coating. An RCA reaction may be performed in the presence of the polylysine coated surface, resulting in simultaneous immobilization and amplification of a nanoball. RCA primers may be supplied in solution or bound to the polylysine-coated surface prior to performing the RCA reaction.

In another example, a streptavidin-coated surface (e.g., beads, flow cells, microwells, or nanowells) may be used to capture nanoballs. In this approach, biotin-linked deoxynucleotides may be incorporated into the nanoballs during RCA. The nanoballs can be bound to the surface by a biotin-streptavidin linkage. A surface may be coated with a streptavidin coating. An RCA reaction may be performed in the presence of the streptavidin coated surface using biotin-linked deoxynucleotides to produce a nanoball that includes biotin moieties resulting in simultaneous immobilization and amplification of the nanoball.

In another embodiment, biotin linked RCA primers may be bound to a surface by a streptavidin-biotin linkage and used to initiate an RCA reaction. A surface may be coated with a streptavidin coating. An oligonucleotide that includes a biotin moiety may be attached to the surface through a biotin-streptavidin linkage. An oligonucleotide may include an RCA primer sequence that is complementary to an RCA primer binding site on a recognition element. An oligonucleotide may be used to capture a recognition element by hybridization of the complementary sequences and initiate the RCA reaction to produce a nanoball. Amplification in the presence of the streptavidin coated surface further anchors the nanoball to the surface.

Following the formation of a nanoball, a determination may be made with respect to the identity of the code. Prior to making the determination, various secondary processing operations are possible within the scope of the assays described herein. The recognition element may include various elements that facilitate secondary processing operations. Examples include restriction endonuclease sites and CRISPR sites.

The nanoball may be converted to double-stranded DNA (dsDNA) prior to fragmentation. The dsDNA nanoball may be fragmented. In one embodiment, the recognition element includes restriction sites which are replicated in the nanoball, and the nanoball is converted to dsDNA and fragmented using a restriction enzyme having specificity for the restriction sites.

Clustered Regularly Interspaced Short Palindromic Repeats (CRISPR) may be used to fragment the nanoball at specific sites.

Random fragmentation of nanoballs may be performed, using existing fragmentation techniques.

Tagmentation may be performed on a dsDNA nanoball, and the tagmentation may be used to add sequencing adapters or other functional sequences to fragments of a dsDNA nanoball.

### Sequencing Preparation

This disclosure provides a variety of techniques for amplifying and preparing circularized recognition elements for sequencing. In certain embodiments, amplification and preparation for sequencing may be performed sequentially (e.g., PCR+primer ligation). In certain embodiments, amplification and preparation for sequencing may be performed in a single reaction (e.g., adapter addition via PCR). Addition of sequencing adapters may be performed with or without RCA amplification of circularized recognition elements.

In one embodiment, sequencing adapters are added via PCR. In this case, amplification and preparation for sequencing may be a single operation. Depending on the recognition element design, the code, UMI, and index may be read in a single operation.

In one embodiment, RCA products (e.g., nanoballs) may be fragmented with restriction endonucleases (RE) that cleave single stranded DNA (e.g., Type II endonucleases, etc.) to yield a multitude of code-containing single stranded nucleic acids. The single-stranded nucleic acids (e.g., the RE reaction products) may then be prepared for sequencing by ligation to adapter sequences.

In one embodiment, sequencing adapters may be added by transposomes that may simultaneously fragment double-stranded DNA and add adapters.

As discussed herein, the assays of the inventive concepts may include a transformation operation. The transformation may involve circularization of a recognition element when a target is present and hybridized to its complementary sequences in the recognition element (e.g., by ligation or gap-fill ligation).

Non-limiting examples of a transformation process of the present disclosure are provided in U.S. application Ser. No. 18/391,323 which is hereby incorporated by reference. In some embodiments, a recognition element includes a UMI sequence, a code, an SBS primer binding sequence, and an index primer binding sequence all situated between a 5′ target end of a recognition element and a 3′ target end of a recognition element. In the presence of a target, the recognition element and the target can hybridize and the hybridized recognition element can be circularized in a ligation reaction to yield a circular modified recognition element. The ligation reaction may be followed by an exonuclease digestion operation to remove unligated recognition elements and targets.

The circular modified recognition element may, in some cases, be amplified in a rolling circle amplification (RCA) to form a nanoball product. For example, in an RCA reaction a SBS primer binding site that is the reverse complement to a SBS primer may be hybridized to a circular modified recognition element and used to initiate the RCA reaction to generate a nanoball. The nanoball is a polymeric molecule (concatemer) that includes multiple repeated copies of a circular modified recognition element, wherein each copy includes a SBS primer binding site, a code, a UMI sequence, target 5′ and 3′ recognition element ends, and an index primer binding site.

In some embodiments, the RCA products (e.g., nanoballs) may be sequenced directly. In some embodiments, sequencing adapters may be added by PCR amplification, which may be followed by clustering and sequencing.

In some embodiments, the sequencing adapters are added to a nanoball for subsequent clustering and sequencing. The PCR reaction may use a pair of amplification primers. Amplification primers may include a sequencing adapter sequence (e.g., a P7 adapter sequence) and an index sequence (e.g., a sample index sequence). Amplification primers may include a second sequencing adapter sequence (e.g., a P5 adapter sequence). Amplification primers are used in a PCR reaction to initiate amplification of a nanoball to generate multiple single probe copies of the nanoball that now include the adapter sequences and the index sequences. Sequencing provides the UMI sequence, the code sequence, and the index sequence.

In another embodiment, the recognition elements of the inventive concepts may include restriction sites. The recognition elements may be designed with restriction sites, or the restriction sites may be added to the recognition elements as part of the assay process. The restriction sites may be amplified and incorporated into the nanoball and provide multiple sites at which to cleave the nanoball into fragments. The digestion products may be further processed for sequencing.

An additional embodiment may include using a primer and polymerase to create RCA products where the entire concatemer is double stranded. This structure can be processed via the restriction endonuclease procedure for restriction endonucleases that cleave double stranded DNA.

Another embodiment may include employing hyperbranched RCA to create many double stranded, code-containing sequences that can be processed via the restriction endonuclease procedure herein.

In certain embodiments, the restriction endonuclease may be a member of the Cas family of proteins or a derivative thereof. These proteins may recognize longer sequences of DNA, making them more specific.

In an additional embodiment, circularized recognition elements may be prepared for sequencing without RCA.

In certain embodiments, the nanoballs of the inventive concepts may be compacted prior to sequencing. Rolling circle amplification may produce linear concatemers of single-stranded DNA. When the substrate for RCA is a circularized recognition element, these concatemers may contain 100s-1000s of copies of a code. When preparing RCA products for sequencing, it may be useful to compact the RCA products. The compacting may produce spherical structures. The compacted structures can increase localization of a signal.

Compaction of RCA products into spherical nanoballs can be accomplished by a variety of techniques. In one embodiment, cationic additives that condense high molecular weight DNA (e.g., spermidine, Mg ions, cationic polymers) may be used. The compactness of a spherical nanoball may be tuned by controlling the concentration of the cationic reagent used. The concentration of the cationic reagent used may be selected to avoid aggregation of multiple nanoballs.

In one embodiment, multivalent oligonucleotide sequences that crosslink sites on RCA products may be used to compact RCA products into spherical nanoballs. The RCA binding sites may be separated by a nucleic acid or polymeric linker to control the degree of compaction. The compactness of the spherical nanoball may, for example, be tuned by controlling the degree of crosslinking in the RCA product.

In one embodiment, incorporation of modified nucleotides followed by crosslinking may be used to compact RCA products into spherical nanoballs. Non-limiting examples of modified nucleotides that may be used include biotinylated nucleotides that bind to streptavidin proteins and nucleotides that covalently react with multifunctional linkers (e.g., amino nucleotides and NHS-terminated linkers). The compactness of the spherical nanoball may, for example, be tuned by controlling the degree of crosslinking in the RCA product.

In certain embodiments, the assays of the inventive concepts may make use of nanopore sequencing. A nanoball or a modified recognition element may be sequenced using nanopore sequencing. Various existing nanopore sequencing sample preparation techniques may be used. Amplification is optional. Various components for other sequencing techniques, such as sequencing primers, may be omitted from the probe. Purification can be accomplished using, for example, SPRI beads, BluePippen or other size selection technologies. Oxford Nanopore Technologies, Inc. (Oxford, UK) provides kits for sample preparation for nanopore sequencing.

In certain embodiments, it may be useful to further amplify RCA products prior to sequencing. For example, in applications that use cell-free DNA (cfDNA) as the input where the analyte number may be low, it may be useful to amplify the RCA product prior to sequencing. In one embodiment, a circle-to-circle amplification approach may be used to produce multiple RCA products from one initial RCA product by monomerization of the concatemer (e.g., cleavage to unit length fragments), recircularization of the unit length fragments (e.g., monomers) and amplification of the newly generated circles in a second RCA reaction to produce multiple RCA product copies for further processing or sequencing. The restriction enzyme approach may be used to digest the initial RCA product to unit length (e.g., monomers). In some cases, an end-to-end joining oligonucleotide plus an end-to-end ligation reaction may be used to circularize the unit size fragments. In some embodiments, the process comprises circularizing and amplifying unit length nanoball fragments to produce multiple RCA nanoball products, as described in U.S. application Ser. No. 18/391,323, which is hereby incorporated by reference.

Non-limiting examples of sequencing techniques suitable for use with the assays disclosed herein include nanopore sequencing, next-generation sequencing, massively parallel sequencing, Sanger sequencing, sequencing by synthesis (SBS), pyrosequencing, sequencing by hybridization, single molecule real-time sequencing, sequencing by oligonucleotide ligation and detection and sequencing by ligation.

In some embodiments, a process for circularizing a recognition element may include a gap-fill ligation reaction that may be used to circularize the recognition element and capture an unknown region of the target that may then be sequenced along with the code.

In some embodiments, an unknown region of a target sequence may be captured by a recognition element transformation reaction and sequenced along with the code. In some embodiments, a recognition element is hybridized to a target and circularized in a gap-fill ligation reaction that captures an unknown region of the target sequence. For example, a recognition element that includes a code (among other elements) and a pair of target recognition elements is hybridized to a target analyte. A target may include a region comprising an unknown sequence. Target recognition elements recognize and hybridize to two or more genomic regions of interest (ROI) of the target simultaneously at sites flanking an unknown region. A gap-fill ligation reaction is performed to copy a region into the recognition element followed by circularizing the recognition element to yield a circular modified recognition element comprising the unknown region of the target. The ligation reaction may be followed by an exonuclease digestion operation to remove unligated linear recognition elements and targets.

In some embodiments, the circular modified recognition element may be amplified in an RCA reaction to form an RCA product comprising multiple copies of the circularized recognition element including multiple copies of the unknown region and the code (among other sequences). The RCA product may be sequenced directly, or sequencing adapters may be added by PCR amplification, followed by clustering and sequencing as described herein.

### Targeted Analyte Assay Workflow

The assays may provide a readout that can be measured alongside the readout of various molecular assays that may be performed in parallel, thereby enabling a multiomic platform for the analysis of different target analytes from a sample.

Non-limiting examples of target analytes include, but are not limited to, proteins, nucleic acids (e.g., DNA and RNA), metabolites, glycosylation, exosomes, viruses, bacteria, and cells (e.g., circulating tumor cells). DNA targets may include reference or wildtype sequences, single nucleotide variants (SNVs), insertion/deletions (indels), copy number variants and methylated nucleotides. An RNA target may be a splice variant.

In one embodiment, an encoded assay may be performed for the analysis of a set of nucleic acid targets from a sample.

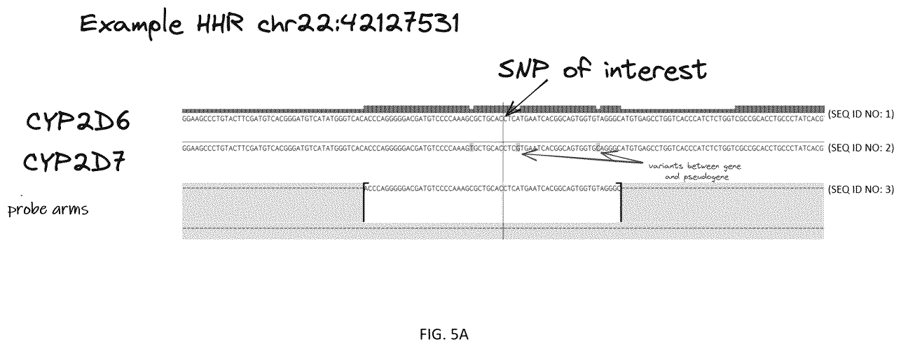

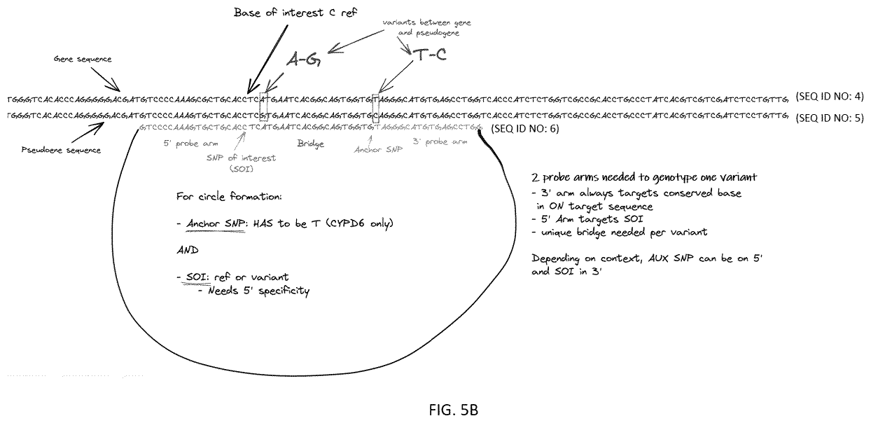

In one embodiment, the analyte is DNA. In an encoded assay, a set of DNA targets may be targeted for detection of a single nucleotide difference relative to a reference nucleotide. A single nucleotide difference may be a change in the methylation status of a nucleotide at a target site of interest. In another example, a single nucleotide difference may be a change in nucleotide usage at a target site of interest, e.g., a single nucleotide polymorphism (SNP), a single nucleotide variant (SNV), or an indel (insertion/deletion). In some embodiments, the set of DNA targets may be targeted for detection of two or more nucleotide differences relative to a reference nucleotide. For example, the multi-region encoded assay may detect two or more SNVs phased from a single DNA molecule. In another example, the multi-region encoded assay may use used to disambiguate genotyping by detecting polymorphisms (e.g., SNP, SNV, indel) in a gene when a corresponding pseudogene (not of interest) is present, such as illustrated in FIGS. 5A-5B. FIG. 5A shows a portion of a gene CYP2D6 (SEQ ID NO: 1) and a high homology pseudogene CYP2D7, when both the gene and the pseudogene have the same variant, however it is a SNP of interest in the gene. One way to maximize detection of the gene and minimize detection of the pseudogene is to look for additional variations between the gene and pseudogene in the area of the SNP of interest. In FIG. 5A, there are three nucleotides that are different in the pseudogene compared to the gene of interest in proximity to the SNP of interest. SEQ ID NO:3 shows exemplary 5′ and 3′ ends of a recognition element, where 5′ end includes one of the three different nucleotides that may be complementary to the gene of interest and not the pseudogene, and 3′ end include the other two nucleotides that may be complementary to the gene or interest and not the pseudogene. As such, while the SNP of interest may hybridize to the end of the recognition element for both the gene and the pseudogene, there may be mismatch of the three different nucleotides between the recognition element and the pseudogene, which may lead to minimal or no hybridization and ligation between 5′ and 3′ ends of the recognition element for the pseudogene, and hence minimal or no amplification and detection of the pseudogene.

FIG. 5B demonstrates another example of a mechanism for detecting a SNP of interest in a gene in the presence of a high homology pseudogene. In FIG. 5B, 5′ probe arm includes the SNP of interest, which is present in both the gene and the pseudogene, whereas the 3′ probe arm includes a nucleotide that will match and hybridize to the gene of interest which is not present in the pseudogene as such no hybridization and subsequent ligation. In this scenario, an additional bridge element is added, wherein one of the nucleotides in the bridge element is complementary to a nucleotide present in the gene of interest which is not present in the pseudogene. It is expected that the gene with no mismatches and homology to 5′ and 3′ ends of the recognition element as well as the bridge element, will result in a ligation of the bridge element to the recognition element ends yielding a circularized recognition element, whereas due to the mismatches with the pseudogene, there may be minimal to no ligation of the bridge element with the ends of the recognition element yielding a non-ligated and circularized recognition element.

In yet another example, the multi-region encoded assay may be used to detect variable versus conserved regions in, for example, 16s rRNA genes of bacterium to identify species and genera of pathogens. Non-limiting examples of bacterial pathogens include food-borne pathogens, sexually-transmitted pathogens, pathogens that cause a disease or a disorder, and so on. In some embodiments the pathogenic infection is or can be caused by Campylobacter, Salmonella, Cellulitis, boils, impetigo, Lyme disease, bacterial vaginosis, Chlamydia, Strep throat, Clostridioides difficile, Escherichia coli.

In one embodiment, the analyte is RNA. In an encoded assay, an RNA sample may, for example, be processed in a reverse transcription reaction to generate cDNA molecules for detection of a set of targets of interest. An encoded RNA assay may, for example, be used to detect and count RNA targets of interest from a sample. In another example, an encoded RNA assay may be used to detect alternative splicing variants for a target of interest.

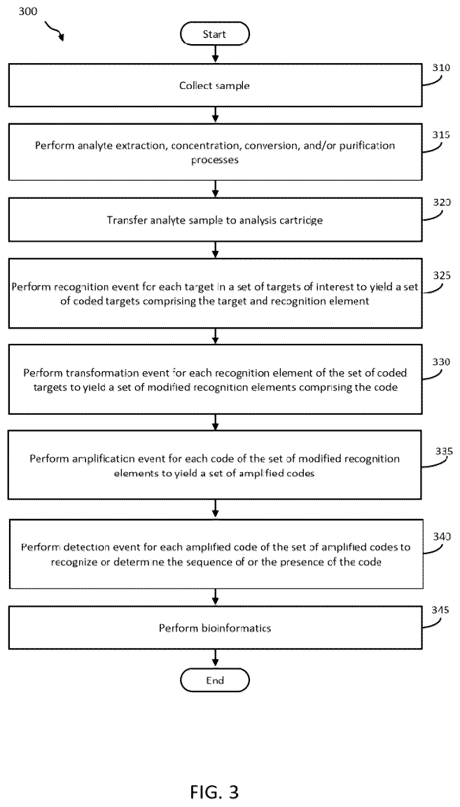

FIG. 3 is an example of a flow diagram of an example of a target analyte assay workflow 300. Assay workflow 300 may include, but is not limited to, the following operations.

At operation 310, a sample may be collected. For example, a blood or saliva sample may be collected. In one example, a whole blood sample may be collected and processed to separate the plasma fraction from the cellular components of whole blood.

At operation 315, analyte extraction, concentration, conversion, and/or purification processes may be performed. In this example, the analyte may be DNA. DNA (e.g., cell-free DNA) in the plasma sample may be extracted, purified, and concentrated for analysis. A proteinase K (ThermoFisher, Waltham, MA) digestion operation may be used to digest proteins present in the plasma sample. In some cases, a heat denaturation operation (e.g., 94-98° C. for 20-30 seconds) may be used to denature double-stranded DNA into single-stranded nucleic acid. A bead-based extraction and concentration protocol may be used to capture single-stranded DNA in the plasma sample. In some embodiments, the bead-based extraction protocol uses magnetically responsive nucleic acid capture beads. The bead-bound DNA may be released from the capture beads using an elution buffer (or other elution means suitable to the capture bead used) to produce a processed DNA sample for analysis.

In one embodiment, the DNA sample may be further processed in a bisulfite conversion reaction for analysis of the methylation status of a set of targets from the sample.

At operation 320, the processed DNA sample may be transferred into an analysis cartridge.

At operation 325, a recognition event for each target in a set of targets may be performed. For example, each target may be uniquely recognized by and hybridized to a recognition element associated with a code (and optionally other elements). In one example, the recognition event for the set of targets may use a panel of multi-region coded recognition elements. In another example, the recognition event for the set of targets may use a panel of multi-region molecular inversion probes. The recognition event may yield a set of coded targets comprising the target and the recognition element.

At operation 330, a transformation event for each recognition element of the set of coded targets may be performed. For example, in the transformation event, a ligation or a gap-fill ligation may produce the modified recognition element, e.g., a version of the recognition element that is ligated or gap-filled. In one example, transformation of a modified recognition element in a ligation or gap-fill ligation reaction may generate a circular molecule. In some cases, an exonuclease cleanup operation may be used following the transformation event to digest any remaining linear single stranded nucleic acids, such as unhybridized coded multi-region recognition elements and single stranded target sequences. The transformation event yields a set of modified recognition elements comprising the code and target sequences or complements thereof.

At operation 335, an amplification event for each of the modified recognition elements may be performed. In one example, the amplification event may be a rolling circle amplification (RCA) reaction to generate a set of target-specific nanoballs that include all the components of the recognition elements. The amplification event may yield a set of amplified recognition elements includes the codes (among other elements).

At operation 340, a detection event for each amplified code of the set of amplified recognition elements may be performed to identify each code. In one example, the code may be decoded by sequencing the recognition element. The detection event may detect the code which is subsequently decoded and used as a proxy for detection of the presence, or absence, of the targeted analyte.

At operation 345, using the code information (and optionally other elements), bioinformatic secondary analysis may be performed on the detection data.

In some embodiments, the amplification event (operation 335) and the detection event (operation 340) may be combined in a single operation.

### Sequencing for Target Detection

In some embodiments, a sequencing library comprising the recognition elements comprising the codes (among other elements) may be generated. The library may be sequenced to identify codes associated with a target of interest. In one embodiment, a sequencing library may be generated from a circularized recognition element (e.g., padlock probe). The padlock probe library may be sequenced to identify the code associated with the target of interest.

### Nanoball Sequencing Library for Target Detection

A sequencing library comprising the recognition element codes (among other elements) may be generated from a set of target-specific nanoballs. The nanoball library may be sequenced to identify codes associated with targets of interest.

In some embodiments, methods for generating a sequencing library from a nanoball may be used to identify the codes associated with the target set of interest comprising preparing the sample preparation (e.g., starting from a whole blood sample, performing the nucleic acid extraction, concentration, and/or purification processes, and transferring the nucleic acid sample to the analysis cartridge).

In some embodiments, the method comprises recognition and transformation events for each target in a set of targets of interest to yield a set of modified recognition elements comprising the code. For example, a set of multi-region coded recognition elements that include target-specific regions associated with a code may be used. The transformation event may include a ligation or a gap-fill ligation reaction to produce a circularized modified recognition element comprising the code. In the transformation event, the multi-region coded recognition element that hybridizes to a target sequence of interest with no mismatches may be ligated to yield a circular modified recognition element comprising the code.

In some embodiments, the method may comprise an amplification event for each recognition element and its associated code of the set of modified recognition elements. For example, a modified recognition element may be amplified in a rolling circle amplification (RCA) to generate a nanoball product.

In some embodiments, the method may comprise a sequencing library that is generated from the nanoball product. For example, 25 cycles of amplification may be used to add sequencing adapters and sample index sequences (among other optional sequences) to the nanoball product generating a sequencing library that includes a set of codes. The sequencing library may be loaded onto a sequencing flow cell (e.g., an Illumina® sequencing flow cell) for next generation sequencing (NGS).

In some embodiments, the method may comprise a detection event for each code of the set of codes. For example, the library may be sequenced using an NGS sequencing protocol to identify the codes (and other elements (e.g., sample index, UMIs)) associated with the set of targets of interest. Direct sequencing on nanoballs for target detection

A set of nanoballs may be directly sequenced to identify codes associated with the set of targets of interest. The code data may then be used as a digital count of the target-specific detection events.

In one embodiment, the nanoballs may be immobilized onto the surface of a sequencing flow cell for direct sequencing on the nanoballs. The nanoballs may be immobilized onto the flow cell surface using an immobilization agent. In one example, the immobilization agent is a surface bound oligonucleotide that is complementary to a sequence on the nanoball. In another example, the immobilization agent is a polypeptide.

To facilitate immobilization of a nanoball on a flow cell surface for direct sequencing, a recognition element associated with a code (e.g., an encoded probe) may include a palindrome sequence that is incorporated into the nanoball to create a secondary structure that compacts (collapses) the nanoball. The compacted nanoball provides a structure that may be more readily directly sequenced.

In some embodiments, the nanoball may be directly sequenced to identify codes associated with the target of interest. In some embodiments, the method comprises preparing the sample. In some embodiments, preparing the samples starts from a whole blood sample, performing the nucleic acid extraction, concentration, and/or purification processes, and transferring the nucleic acid sample to the analysis cartridge.

In some embodiments, the method may comprise recognition and transformation events for each target in a set of targets of interest to yield a set of modified recognition elements comprising the code. For example, a set of multi-region coded recognition elements that include target-specific recognition regions associated with a code may be used. The transformation event may include a ligation or a gap-fill ligation reaction to produce a circularized modified recognition element comprising the code. In the transformation event, the coded multi-region recognition elements that hybridize to a target sequence of interest with no mismatches may be ligated to yield a circular modified recognition element comprising the code.

In some embodiments, the method may comprise an amplification event for each of the modified recognition elements comprising a code. For example, a modified recognition element may be amplified in a rolling circle amplification (RCA) to generate a nanoball product.

In some embodiments, the method may comprise a nanoball product that is loaded onto the surface of a sequencing flow cell. For example, a nanoball product is loaded onto an Illumina® flow cell, such as a MiSeq flow cell. The nanoballs may be immobilized onto the flow cell surface using an immobilization agent. In one example, the immobilization agent is a surface bound oligonucleotide that is complementary to a sequence on the nanoball. In another example, the immobilization agent is a polypeptide.

In some embodiments the method may comprise sequencing for each nanoball and its amplified code. For example, the nanoball is directly sequenced to identify codes associated with the set of targets of interest. The code data may then be used as a digital count of the target.

### Methylation Assays

Assays of the inventive concepts may be used to interrogate a methylation status of a target sequence of interest at more than one region of the target. In one embodiment, methylated cytosines in a target sequence of interest may be detected using assays that include a conversion reaction to detect methylated cytosines. In another embodiment, methylated cytosines in a target sequence of interest may be detected using assays that do not use a conversion reaction (e.g., conversion-free).

In one embodiment of a conversion assay for detection of methylated cytosines, a bisulfite conversion reaction that converts non-methylated cytosines to thymine (C→T) may be used.

For example, a methylated cytosine assay using encoded probes may include: (i) a bisulfite conversion reaction to convert non-methylated cytosine to thymine (C→T); (ii) a recognition event, in which a target nucleic acid is uniquely recognized and bound by a recognition element associated with a code (e.g., an encoded probe); (ii) a transformation event, in which a molecular transformation of the recognition element produces a modified recognition element comprising the code; and (iii) a detection event, that uses the code as a proxy for detection of the target nucleic acid, e.g., by recognizing and decoding the code or by sequencing (and optionally other elements).

In some embodiments, a methylated target site of interest may be interrogated using an encoded recognition element in combination with a transformation event that includes a ligation reaction to detect the methylation status of the target site.

In one embodiment, the recognition element (e.g., an encoded probe) may be a coded multi-region recognition element that includes a 3′-terminal guanine (“G”). The transformation event (e.g., ligation) to generate the modified recognition element may occur when 3′-guanine is matched to a cytosine at a target site of interest and hybridization occurs.

Aspects disclosed herein provide methods for using a bisulfite conversion reaction in combination with a coded multi-region recognition element to detect a methylated target site of interest. In this example, a DNA sample may include a target sequence of interest that may be methylated or unmethylated at a CpG site of interest. A bisulfite conversion reaction is used to convert non-methylated cytosine to thymine (C→T) in the target sequence. In this example the target sequence is methylated at two cytosines.

In the recognition event, the target sequence may be recognized and bound by a recognition element (e.g., padlock probe) associated with a code. The padlock probe may have two recognition elements, each with a 3′-terminal G nucleotide that base pairs with the target C at the CpG sites of interest.

In the transformation event, ligation of multi-region padlock recognition element may occur when both of 3′-termini of the recognition element of the padlock probe (e.g., a guanine “G”) are matched to the target site “C” of interest in the target sequence to generate a circularized modified padlock recognition element. No ligation may occur at the target site “T” in the bisulfite converted target sequence as the recognition element does not hybridize to the target sequence and ligation does not occur. The modified and ligated padlock recognition element may be amplified in an RCA reaction to generate a nanoball product comprising many copies of the code (among other elements) and the code may be detected and decoded, or sequenced.

In one embodiment, the recognition element (e.g., a molecular inversion probe) may be designed to target two methylated cytosine sites of interest in a target sequence of interest. A gap-fill ligation event using all dNTPs may be used to generate the modified recognition element comprising the code. In this approach, both methylated cytosines may be present in the target nucleic acid molecule for ligation to occur. The requirement for multiple matches has several advantages: (i) it provides enhanced specificity relative to a single match at a methylated cytosine; (ii) the ability to discriminate between a disease state (e.g., all CpG sites in a region are methylated) and a healthy state (e.g., some CpG sites are methylated) is increased by requiring multiple methylated cytosines for detection; and (iii) multiple matches can be used to correct for incomplete bisulfite conversion of unmethylated cytosines at the target site of interest.

In some embodiments, a DNA sample may include a target sequence of interest that may be methylated at multiple CpG sites. In some embodiments, a bisulfite conversion reaction is used to convert non-methylated cytosine to thymine (C→T) in the target sequence. In some embodiments, in the recognition event, a target sequence is recognized and bound by a recognition element associated with a code, e.g., multi-region molecular inversion probe. A multi-region molecular inversion probe includes a 3′-probe arm that terminates at a first methylated cytosine site and a 5′-probe arm that terminates at a second methylated cytosine site. Both a 3′-GC match and a 5′-GC match during the recognition event (hybridization) may be included for a transformation event to occur. In some embodiments, in the transformation event, a gap-fill ligation reaction using all dNTPs is performed. The 3′-GC match may be included for polymerase extension in the gap-fill reaction. The 5′-GC match may be included for ligation of the gap-filled molecule. Gap-fill ligation generates a circularized modified recognition element. If no incorporation of dGTP occurs at the target site “T” in the bisulfite converted target sequence then no transformation to a circular modified recognition element occurs (e.g., a non-methylated target sequence). In some embodiments, the circular modified recognition element may be amplified in an RCA reaction to generate a nanoball product comprising many copies of the recognition element and its code (among other elements) and the code may be detected and decoded or sequenced.

### Genotyping Assays

The assays of the inventive concepts may be used in a genotyping assay. A target site of interest may be interrogated using an encoded recognition element in combination with a ligation reaction to detect a polymorphism such as a single nucleotide variant (SNV) of interest. In one example, the polymorphism may be a single nucleotide polymorphism (SNP).

In one embodiment, a genotyping assay using encoded recognition elements may include: (i) a recognition event, in which a target nucleic acid is uniquely recognized and hybridized to a recognition element associated with a code (e.g., a multi-region encoded probe); (ii) a transformation event, in which a molecular transformation of the recognition element produces a modified recognition element comprising the code; and (iii) a detection event, that uses the detected code as a proxy for detection of the target nucleic acid, e.g., by recognizing or decoding the code (and optionally other elements).

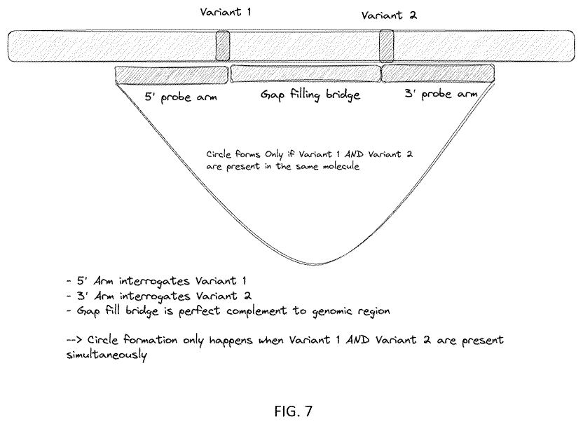

In one embodiment, the recognition element may be a multi-region encoded recognition element that includes a 3′ probe arm that has a 3′-terminal nucleotide that is matched to a polymorphism of interest, and a 5′ probe arm that has a 5′ terminal nucleotide that is matched to another polymorphism of interest. The transformation event (e.g., ligation) to generate the modified recognition element may occur when 3′-nucleotide is matched to the polymorphism at the target site of interest. FIG. 7 provides a non-limiting example of a multi-region recognition element that may be used to interrogate a first genomic ROI (“variant 1”) and a second genomic ROI (“variant 2”) simultaneously, where the first genomic ROI and the second genomic ROI are phased variants from a single genomic locus.

While, in this example, two variants of interest are detected, the multi-region encoded recognition element may be designed to detect more than two polymorphisms of interest. For example, 3′ probe arm and 5′ probe arm may have multiple nucleotides that match a plurality of polymorphisms of interest. In some embodiments, the multi-region encoded recognition element can detect greater than or equal to 2, 3, 4, 5, 6, 7, 8, 9, 10, or more polymorphisms. In some embodiments, the polymorphisms comprise a SNP, a SNV, a CNV, or an indel.

In one embodiment, the recognition element may be a molecular inversion probe that includes a 3′-probe arm having a 3′-terminal single base gap at a target site of interest, and a 5′-probe arm. A gap-fill ligation event using a single added nucleotide for each target site of interest may then be used to generate the modified recognition element comprising the code when the corresponding nucleotide is incorporated.

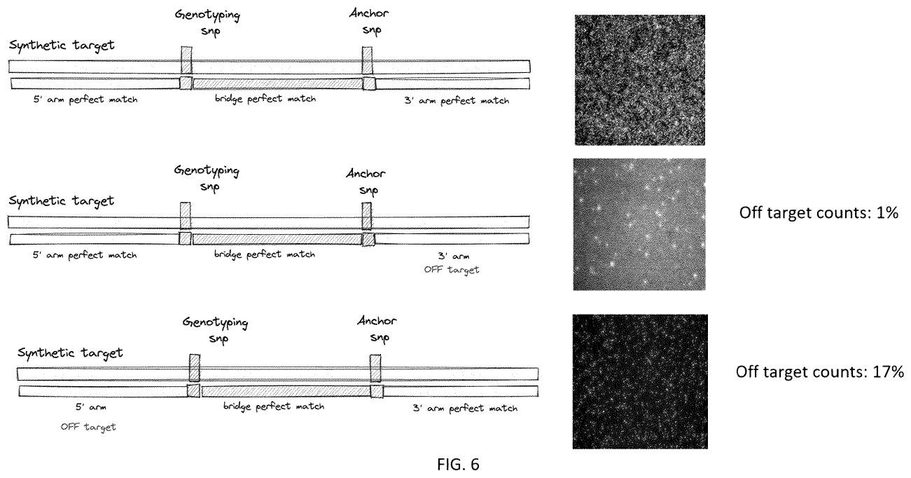

In some embodiments, the recognition of both of the target sites of interest results (e.g., a perfect match) in the highest density, size and uniformity of DNA nanoballs (FIG. 6 top) relative to multi-region recognition elements and synthetic targets nucleic acid sequences having at least one off-target match (FIG. 6, middle and).

### RNA Assays

The assays of the inventive concepts may be used in an RNA analysis assay.

In one embodiment, an RNA assay using encoded recognition elements may include:
(i) a reverse transcription reaction to convert RNA (e.g., polyA RNA) to cDNA; (ii) a recognition event, in which a target cDNA is uniquely recognized and hybridized to a recognition element associated with a code (e.g., a multi-region encoded recognition element);
(ii) a transformation event, in which a molecular transformation of the recognition element produces a modified recognition element comprising the code; and (iii) a detection event, that uses the code as a proxy for detection of the target RNA, e.g., by recognizing or decoding the code (and optionally other elements).

In some cases, the reverse transcription operation (i) may be omitted and a ligase which will ligate single stranded DNA in a DNA: RNA hybrid may be used in the transformation event. In one example, the ligase is a PBCV-1 DNA ligase. In another example, the ligase is a Chlorella virus DNA ligase.

In one embodiment, the encoded recognition element may be a padlock probe that includes a recognition element associated with a code.

In one embodiment, the encoded recognition element may be a molecular inversion probe that includes a recognition element associated with a code.

Assays of the inventive concepts may be used to detect and count RNA derived targets of interest in a sample.

Assays of the inventive concepts may be used to detect alternative splicing variants for a target of interest. In one example, splicing variants may be identified by placing one half of a recognition element (e.g., a multi-region coded recognition element) on either side of a splice junction. The transformation event (e.g., ligation) to generate the modified recognition element may occur when 3′-nucleotide of the recognition element is matched and hybridizes to the splice variant at the target site of interest.

In another example, splice variants may be identified using a molecular inversion probe comprising a code and an extension ligation reaction, wherein one probe arm spans the splice junction of interest.

### Samples

Non-limiting examples of tissues from which nucleic acids may be extracted include, but are not limited to, solid tissue, lysed solid tissue, fixed tissue samples, whole blood, plasma, serum, dried blood spots, buccal swabs, forensic samples, fresh or frozen tissue, biopsy tissue, organ tissue, cultured or harvested cells, and bodily fluids.

In various embodiments, a sample may include a biological sample, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes.

Samples may be provided directly from biological sources, or may be processed samples, such as samples which are enriched for targets, nucleic acids, or proteins from any of the foregoing sources.

### Targets

The assays provide a readout that can be measured alongside the readout of various molecular assays that may be performed in parallel, thereby enabling a multiomic platform for the analysis of different target analytes from a sample. Examples of target analytes include, but are not limited to, proteins, nucleic acids (e.g., DNA and RNA), metabolites, glycosylation, exosomes, viruses, bacteria, and cells (e.g., circulating tumor cells). DNA targets include, but are not limited to, polymorphisms such as single nucleotide variants (SNVs), single nucleotide polymorphisms (SNPs) insertion/deletions (indels), copy number variations, wildtype sequences, and methylated nucleotides. An RNA target may be a splice variant.

Targets may include any biological markers. Examples include, but are not limited to, biological markers for screening or diagnosing cancer. In one embodiment, targets include a panel of methylation markers for diagnosing cancer. Non-limiting examples of panels of markers which may be targeted can be found in WO2019195268, entitled “Methylation markers and targeted methylation probe panels,” and WO2020069350A1, entitled “Methylation markers and targeted methylation probe panel,” the entire disclosures of which (including without limitation the sequence listings) are incorporated herein by reference. Targets may be obtained from biopsies, circulating nucleic acid samples, or nucleic acids from other samples.

In one embodiment, targets include a panel of single nucleotide variants (SNVs) or single nucleotide polymorphisms (SNPs) for diagnosing cancer.

The multi-region recognition elements disclosed herein are also useful for detecting pathogens by detecting the variable versus conserved regions in 16s rRNA genes of a pathogen of interest.

The multi-region recognition elements disclosed herein are also useful for disambiguating genes from pseudogenes. See FIGS. 5A-5B.

### Diagnostics and Screening

The methods of the inventive concepts may be used for screening or diagnosing a subject for a disease, such as cancer or for selecting a therapy for treating a disease, such as selecting a therapy for treating a cancer. The methods of the inventive concepts may be used for monitoring and managing a therapeutic regimen for treatment efficacy and potential adjustment.

In one embodiment, the methods of the inventive concepts may be used in a liquid biopsy application. In one example, a liquid biopsy assay may include determination of the methylation status and/or the variant usage of a set of target sequences.

In one embodiment, the methods of the inventive concepts may be used in a pathogen detection application. In one example, pathogen detection may include detecting both a protein and nucleic acid (e.g., an RNA) associated with the pathogen.

In one embodiment, the methods of the inventive concepts may be used to monitor and/or determine complications associated with a transplantation procedure.

In another embodiment, the methods of the inventive concepts may be used to detect short nucleic acid fragments. In some embodiments, the nucleic acid fragments are DNA, cDNA, or RNA. The nucleic acid fragments may be extracted from a cell by human manipulation of the cell or sample processing (e.g., cell membrane disruption, lysis, vortex, shearing, etc.). In some embodiments, nucleic acid fragments are circulating cell-free nucleic acids. The cell-free nucleic acids may be produced in a cell and released from the cell by physiological means, including, e.g., apoptosis, and non-apoptotic cell death, necrosis, autophagy, spontaneous release (e.g., of a DNA/RNA-lipoprotein complex), secretion, and/or mitotic catastrophe. The cell-free nucleic acid may be released from a cell by a biological mechanism, (e.g., apoptosis, cell secretion, vesicular release

### II. SYSTEMS

### 1. Sequencing Systems

In some embodiments, the systems may comprise a solid substrate. In some embodiments, the systems may comprise a welled plate or a flowcell. In some embodiments, the systems may comprise a fluid flow controller, a temperature controller, an imaging system, a computer system, or any combination thereof.

In some embodiments, the systems may include a solid substrate or a solid surface. The solid substrate or surface may be referred to as a substrate, a support, a solid support, or a surface. The substrate may be modified for immobilizing recognition elements or concatemeric amplification products, or both. Non-limiting examples of solid substrates include, but are not limited to, glass, modified or functionalized glass, plastics, polysaccharides, nylon, nitrocellulose, ceramics, resins, silica, silica-based materials, carbon, metals, inorganic glasses, plastics, optical fiber bundles, optically clear glass, and other polymers. In some embodiments, the plastic solid substrate may include acrylics, polystyrene and copolymers of styrene and other materials, polypropylene, polyethylene, polybutylene, or polyurethanes. In some embodiments, the silica-based solid substrate may include silicon or modified silicon.

In some embodiments, the substrate may be a welled plate. In some embodiments, the substrate may be a 96-well plate. In some embodiments, the substrate may be a 4-well plate, a 6-well plate, an 8-well plate, a 12-well plate, a 24-well plate, a 48-well plate, a 384-well plate, an 864-well plate, or a 1,536-well plate. In some embodiments, the substrate may have greater than or equal to 96 wells. In some embodiments, the substrate may have less than or equal to 96 wells.

In some embodiments, the substrate may be a flowcell. In some embodiments, the flowcell may have two or more lanes. In some embodiments, the flowcell may have two or less lanes.

In some embodiments, the substrate may be a microarray, a slide, a chip, a microwell, a tube, a column, a particle, a bead, or a paramagnetic bead.

In some embodiments, the substrate may comprise a coating. In some embodiments, the coating may comprise a layer that may be charged. In some embodiments, the coating layer may be positively charged. In some embodiments, the coating layer may be negatively charged. In some embodiments, the coating may be non-charged. In some embodiments, the substrate may comprise a surface comprising a cation-coating layer. In some embodiments, the substrate may comprise a surface comprising an anion-coating layer. In some embodiments, the substrate may comprise a surface comprising a neutral-charged layer. In some embodiments, the substrate may be coated with streptavidin. In some embodiments, the substrate may be coated with avidin. In some embodiments, the substrate may be coated with one or more antibodies.

The systems disclosed herein may comprise a fluidics system. The fluidics system may comprise a fluid flow controller. In some embodiments, the fluid flow controller may comprise one or more pumps, valves, mixing manifolds, reagent reservoirs, waste reservoirs, or any combination thereof. In some embodiments, the fluidic system and subcomponents of the fluidics system are fluidically connected to the reaction vessel of the present disclosure. In some embodiments, the reaction vessel comprises a solid substrate configured to immobilize the recognition elements or concatemeric amplification products thereof.

The systems disclosed herein may comprise a temperature system. The temperature system may comprise a temperature controller. The temperature controller may be incorporated into the systems described herein to facilitate accuracy of the methods and systems described herein. In some embodiments, the temperature controller may comprise temperature control components. Non-limiting examples of temperature control components include resistive heating elements, infrared light sources, heating or cooling devices, heat sinks, thermocouples, thermistors, or a combination thereof. In some embodiments, the temperature controller may provide changes in temperature over specified time intervals. In some embodiments, the temperature controller may provide an increase in temperature. In some embodiments, the temperature controller may provide a decrease in temperature. In some embodiments, the temperature controller may provide for cycling of temperatures between two or more set temperatures so that thermocycling or amplification may be performed. In some embodiments, the temperature controller may provide a constant temperature.

The systems disclosed herein may comprise an imaging system. In some embodiments, signals produced by the labeled probes disclosed herein may be imaged by the imaging systems disclosed herein. The imaging system may comprise one or more light sources, one or more optical components, one or more filters, one or one or more imaging sensors for imaging and detection, or a combination thereof. In some embodiments, the one or more light sources may comprise light from a bulb. In some embodiments, the one or more optical components may comprise lenses, mirrors, digital mirror devices, prisms, optical filters, colored glass filters, narrowband interference filters, broadband interference filters, dichroic reflectors, diffraction gratings, apertures, optical fibers, optical waveguides, or a combination thereof. In some embodiments, the one or more imaging sensors may comprise a charge-coupled device (CCD) sensor or camera, a complementary metal-oxide-semiconductor (CMOS) imaging sensor or camera, a negative-channel metal-oxide semiconductor (NMOS) imaging sensor or camera, or a combination thereof.

### 2. Computer Systems

Aspects disclosed herein, in some embodiments, provide a system comprising a computer processor and an electrowetting cartridge. The computer processor may be programmed to execute any one of the methods disclosed herein.

Aspects disclosed herein, in some embodiments, provide a system for conducting an assay for a set of targets or target analytes. The system may comprise a reaction vessel. The system may comprise a reagent dispensing module. The system may comprise a software to execute any of the methods disclosed herein. The system may execute the methods disclosed herein robotically.

Aspects disclosed herein, in some embodiments, provide a computer system for detecting two or more target fragments. The system may comprise a non-transitory memory. The system may comprise a processor in communication with the non-transitory memory. The processor may be configured to execute the following operations in order to effectuate a method. The method may comprise providing a synthetic oligonucleotide scaffold comprising a 5′ region and a 3′ region. The method may comprise providing a coded recognition element comprising. The coded recognition element may comprise a 5′ probe arm and a 3′ probe arm. The 5′ probe arm may have a first region complementary to 3′ region of the synthetic oligonucleotide scaffold. The 3′ probe arm may have a second region complementary to a 5′ region of the target. The coded recognition element may comprise a soft decodable code. The soft decodable code may comprise at least one segment encoding one or more symbols that correspond to a sequence of the coded recognition element. The method may comprise providing a one or more bridge elements comprising a nucleic acid sequence that is complementary to a region of the synthetic oligonucleotide scaffold interposed between 5′ region and 3′ region of the coded recognition element. The method may comprise introducing a sample comprising the two or more target fragments to the synthetic oligonucleotide scaffold, the coded recognition element, and the bridge element. The method may comprise conditions sufficient to form a nucleic acid complex. The method may comprise subjecting the nucleic acid complex to a molecular transformation event in the presence of the two or more target fragments to yield a modified recognition element comprising the soft decodable code. The method may comprise performing an amplification event of the modified recognition element comprising the soft decodable code. The method may comprise detecting the two or more target fragments associated with the modified recognition element by decoding the amplified soft detectable code.

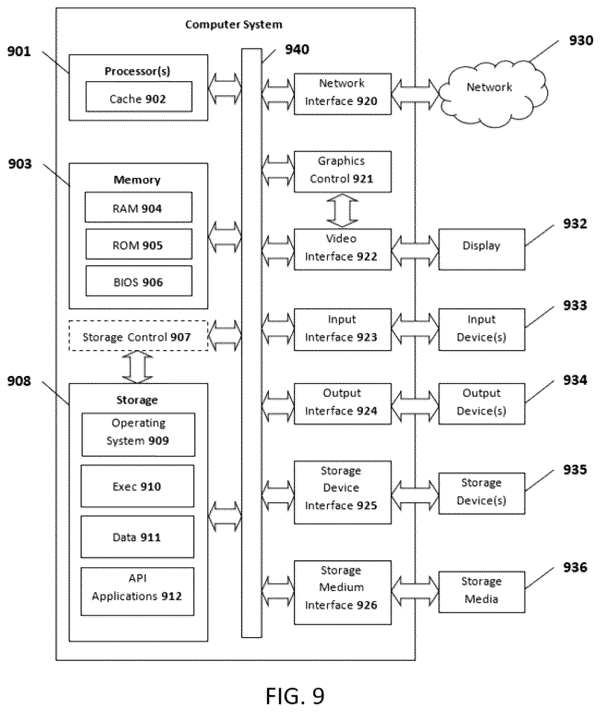

Disclosed herein, in some embodiments, are methods and systems of the present disclosure utilizing one or more computer systems. Referring to FIG. 9, an example of a block diagram is shown depicting an example machine that includes a computer system 900 (e.g., a processing or computing system) within which a set of instructions can execute for causing a device to perform or execute any one or more of the aspects and/or methodologies. The components in FIG. 9 are examples and do not limit the scope of use or functionality of any hardware, software, embedded logic component, or a combination of two or more such components implementing particular embodiments.

Computer system 900 may include one or more processors 901, a memory 903, and a storage 908 that communicate with each other, and with other components, via a bus 940. The bus 940 may also link a display 932, one or more input devices 933 (which may, for example, include a keypad, a keyboard, a mouse, a stylus, etc.), one or more output devices 934, one or more storage devices 935, and various tangible storage media 936. All of these elements may interface directly or via one or more interfaces or adaptors to the bus 940. For instance, the various tangible storage media 936 can interface with the bus 940 via storage medium interface 926. Computer system 900 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 900 may include one or more processor(s) 901 (e.g., central processing units (CPUs), general purpose graphics processing units (GPGPUs), or quantum processing units (QPUs)) that carry out functions. Processor(s) 901 may optionally contain a cache memory unit 902 for temporary local storage of instructions, data, or computer addresses. Processor(s) 901 are configured to assist in execution of computer readable instructions.

Computer system 900 may provide functionality for the components depicted in FIG. 9 as a result of the processor(s) 901 executing non-transitory, processor-executable instructions embodied in one or more tangible computer-readable storage media, such as memory 903, storage 908, storage devices 935, and/or storage medium 936. The computer-readable media may store software that implements particular embodiments, and processor(s) 901 may execute the software. Memory 903 may read the software from one or more other computer-readable media (such as mass storage device(s) 935, 936) or from one or more other sources through a suitable interface, such as network interface 920. The software may cause processor(s) 901 to carry out one or more processes or one or more steps of one or more processes described or illustrated herein. Carrying out such processes or steps may include defining data structures stored in memory 903 and modifying the data structures as directed by the software.

The memory 903 may include various components (e.g., machine readable media) including, but not limited to, a random-access memory component (e.g., RAM 904) (e.g., static RAM (SRAM), dynamic RAM (DRAM), ferroelectric random-access memory (FRAM), phase-change random access memory (PRAM), etc.), a read-only memory component (e.g., ROM 905), and any combinations thereof. ROM 905 may act to communicate data and instructions unidirectionally to processor(s) 901, and RAM 904 may act to communicate data and instructions bidirectionally with processor(s) 901. ROM 905 and RAM 904 may include any suitable tangible computer-readable media described herein. In one example, a basic input/output system 906 (BIOS), including basic routines that help to transfer information between elements within computer system 900, such as during start-up, may be stored in the memory 903.

Fixed storage 908 may be connected bidirectionally to processor(s) 901, optionally through storage control unit 907. Fixed storage 908 may provide additional data storage capacity and may also include any suitable tangible computer-readable media described herein. Storage 908 may be used to store operating system 909, executable(s) 910, data 911, applications 912 (application programs), and the like. Storage 908 can also include an optical disk drive, a solid-state memory device (e.g., flash-based systems), or a combination of any of the above. Information in storage 908 may, in appropriate cases, be incorporated as virtual memory in memory 903.

In one example, storage device(s) 935 may be removably interfaced with computer system 900 (e.g., via an external port connector (not shown)) via a storage device interface 925. Particularly, storage device(s) 935 and an associated machine-readable medium may provide non-volatile and/or volatile storage of machine-readable instructions, data structures, program modules, and/or other data for the computer system 900. In one example, software may reside, completely or partially, within a machine-readable medium on storage device(s) 935. In another example, software may reside, completely or partially, within processor(s) 901.

Bus 940 may connect a wide variety of subsystems. Herein, reference to a bus may encompass one or more digital signal lines serving a common function, where appropriate. Bus 940 may be any of several types of bus structures including, but not limited to, a memory bus, a memory controller, a peripheral bus, a local bus, and any combinations thereof, using any of a variety of bus architectures. As an example and not by way of limitation, such architectures may include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 900 may also include an input device 933. In one example, a user of computer system 900 may enter commands and/or other information into computer system 900 via input device(s) 933. Examples of an input device(s) 933 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 933 may be interfaced to bus 940 via any of a variety of input interfaces 923 (e.g., input interface 923) including, but not limited to, serial, parallel, game port, USB, FIREWIRE, THUNDERBOLT, or any combination of the above.

In particular embodiments, when computer system 900 is connected to network 930, computer system 900 may communicate with other devices, specifically mobile devices and enterprise systems, distributed computing systems, cloud storage systems, cloud computing systems, and the like, connected to network 930. Communications to and from computer system 900 may be sent through network interface 920. For example, network interface 920 may receive incoming communications (such as requests or responses from other devices) in the form of one or more packets (such as Internet Protocol (IP) packets) from network 930, and computer system 900 may store the incoming communications in memory 903 for processing. Computer system 900 may similarly store outgoing communications (such as requests or responses to other devices) in the form of one or more packets in memory 903 and communicated to network 930 from network interface 920. Processor(s) 901 may access these communication packets stored in memory 903 for processing.

Examples of the network interface 920 include, but are not limited to, a network interface card, a modem, and any combination thereof. Examples of a network 930 or network segment 930 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 930, may employ a wired and/or a wireless mode of communication. In general, any network topology may be used.

Information and data can be displayed through a display 932. Examples of a display 932 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 932 can interface to the processor(s) 901, memory 903, and fixed storage 908, as well as other devices, such as input device(s) 933, via the bus 940. The display 932 is linked to the bus 940 via a video interface 922, and transport of data between the display 932 and the bus 940 can be controlled via the graphics control 921. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 932, computer system 900 may include one or more other peripheral output devices 934 including, but not limited to, an audio speaker, a printer, a storage device, and any combinations thereof. Such peripheral output devices may be connected to the bus 940 via an output interface 924. Examples of an output interface 924 include, but are not limited to, a serial port, a parallel connection, a USB port, a FIREWIRE port, a THUNDERBOLT port, and any combinations thereof.

In addition, or as an alternative, computer system 900 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system may be, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google Android®, Microsoft® Windows Phone® OS, Microsoft Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

a. Non-Transitory Computer Readable Storage Medium

In some embodiments, the systems and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

b. Computer Program

In some embodiments, the systems and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

c. Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, XML, and document oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous JavaScript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft Silverlight®, Java™, and Unity®.

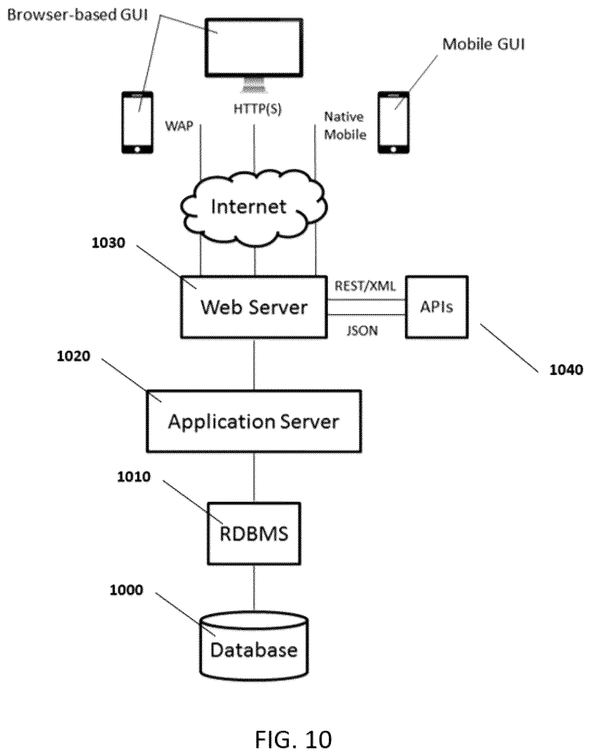

Referring to FIG. 10, in a particular embodiment, an application provision system may comprise one or more databases 1000 accessed by a relational database management system (RDBMS) 1010. Suitable RDBMSs include, but are not limited to, Firebird, MySQL, PostgreSQL, SQLite, Oracle Database, Microsoft SQL Server, IBM DB2, IBM Informix, SAP Sybase, Teradata, and the like. In this embodiment, the application provision system may further comprise one or more application severs 1020 (such as Java servers, .NET servers, PHP servers, and the like) and one or more web servers 1030 (such as Apache, IIS, GWS and the like). The web server(s) optionally expose one or more web services via app application programming interfaces (APIs) 1040. Via a network, such as the Internet, the system provides browser-based and/or mobile native user interfaces.

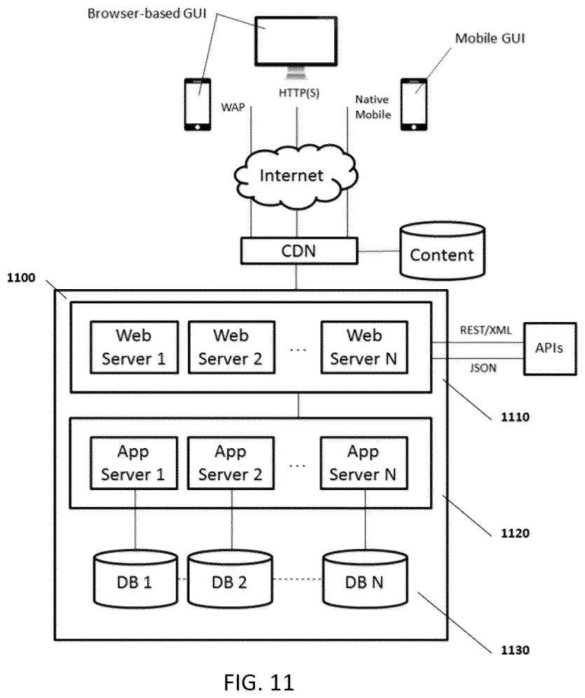

Referring to FIG. 11, in a particular embodiment, an application provision system alternatively has a distributed, cloud-based architecture 1100 and comprises elastically load balanced, auto-scaling web server resources 1110 and application server resources 1120 as well synchronously replicated databases 1130.

d. Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung Apps, and Nintendo® DSi Shop.

e. Standalone Application

In some embodiments, a computer program may include a standalone application, which may be a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

f. Web Browser Plug-In

In some embodiments, the computer program includes a web browser plug-in (e.g., extension, etc.). In computing, a plug-in is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) may be software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

g. Software Modules

In some embodiments, the systems and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

h. Databases

In some embodiments, the systems and methods disclosed herein include one or more databases, or use of the same. In view of the disclosure provided herein, those of skill in the art will recognize that many databases are suitable for storage and retrieval of information. In various embodiments, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing-based. In a particular embodiment, a database is a distributed database. In other embodiments, a database is based on one or more local computer storage devices.

i. Soft Decision Decoding

In some embodiments, the systems disclosed herein may include use of decoding. In some embodiments, decoding may make use of a hard decision decoding model. In another embodiment, decoding may make use of a soft decision decoding model.

For soft decision decoding, it may not be necessary to identify each base specifically. For example, signals generated during each detection event may be detected and recorded to produce a data set that may be used as input into a model to calculate a probability that a specific code is present without requiring that each base of a code be determined. Although it may not be necessary in a soft decision decoding model to make a hard decision about the identity of each nucleotide, a model may nevertheless include assigning a probability or identity to each nucleotide in the sequence of a code, wherein each nucleotide in the sequence of a code may be sequenced. Data gathered includes intensity readings for signals produced by the hybridized detection polynucleotide fluorescent moiety in various spectral bands. A set of intensity readings are detected by imaging, stored and used as input into a soft decision decoding model for determining a probability that a particular code is present, and hence a target nucleic acid is present in the sample.

A model may be developed or trained using data from known codes, such as signal intensity data across a predetermined spectrum. The model may be used to calculate a set of probabilities across a set of one or more codes, indicating, for example, for each code, a probability that it is present in a concatemeric amplification product.

The probability that a particular code is present may be indicative of the probability that a particular target molecule associated with the code is present in the sample of interest. Data indicating the probability that a particular target is present is, for example, to calculate probabilities relevant to diagnosis or screening of various medical conditions, or selection of drugs for treatment of various medical conditions.

A soft decoding decision model may include using an algorithm to predict the presence of target molecules from a sample. In some embodiments, the algorithm is a soft-decision decoding algorithm. In some embodiments, the algorithm is applied to the codes of the concatemeric amplification products for predicting the presence of a target molecule from a sample.

The systems disclosed herein may comprise soft decision decoding to predict the presence of the code in a recognition element or concatemeric amplification product thereof, wherein the presence of the code correlates and serves as a proxy for the presence of a target nucleic acid in a sample. In some embodiments, the methods described herein may use soft decision decoding. In some embodiments, the methods described herein may use hard decision decoding. For hard decision decoding, signals from queried concatemers may be extracted from images. This may be the same for soft decision decoding, in that signals that are generated and imaged are extracted from the images. For hard decision decoding, hard basecalls may be generated from the intensities of the signals, whereas with soft decision decoding no hard basecalls are necessary as all of the signal range is retained. The code assignment for hard decision decoding is determined by matching nucleotide reads to codes, whereas with soft decision decoding, the signals are cross correlated against the expects signals and the most likely code is assigned, as such a probabilistic methodology. When using soft decision decoding, it may not be necessary for the model to identify each base specifically. For example, signals (e.g., fluorescent signals) generated during each cycle of a detection process may be detected and recorded to produce a data set that may be used as input into a model to calculate a probability that a specific code is present.

### III. KITS

Provided herein are kits related to the methods, compositions and systems described herein. In some embodiments, the kits may comprise a plurality of recognition elements, one or more buffers, one or more reagents, instructions for use, a manual, a protocol, or a combination thereof.

In some embodiments, a kit may comprise one or more buffers. In some embodiments, a kit may comprise two or more buffers. In some embodiments, a first buffer of a kit may be configured to promote hybridization. In some embodiments, a second buffer of a kit may be configured to promote de-hybridization, ligation, nucleic acid digestion, storage of a purified molecule. In some embodiments, a kit may comprise one or more reagents. In some embodiments, a kit comprises one or more enzymes. In some embodiments, a kit comprises one or more of a ligase, a DNA polymerase, and an exonuclease. In some embodiments, a kit may comprise instructions for use, a manual, a protocol, or a combination thereof. In some embodiments, a kit may comprise one or more 96 well plates. In some embodiments, one of the 96 well plates of a kit may be configured to be assayed by an optical imaging device described herein.

### IV. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term “about” means approximately, roughly, around, or in the region of. The term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range. For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other existing factors depending on the properties sought to be obtained by the presently disclosed subject matter.

The term “and” is used interchangeably with “or” unless expressly stated otherwise.

The terms “include”, “including”, and the like are to be construed in an inclusive sense as opposed to an exclusive or exhaustive sense; that is to say, in the sense of “including, but not limited to.”

The terms “coded” and “encoded” are intended to have the same meaning and are herein used interchangeably.

The term “linked” with respect to two nucleic acids means a fusion of a first moiety to a second moiety at the C-terminus or the N-terminus, but also includes insertion of the first moiety to the second moiety into a common nucleic acid. Thus, for example, the nucleic acid A may be linked directly to nucleic acid B such that A is adjacent to B (-A-B-), but nucleic acid A may be linked indirectly to nucleic acid B, by intervening nucleotide or nucleotide sequence C between A and B (e.g., -A-C-B- or -B-C-A-). The term “linked” is intended to encompass these various possibilities.

The terms “optimum”, “optimal”, “optimize”, and the like are not intended to limit the inventive concepts to the absolute optimum state of the aspect or characteristic being optimized but will include improved but less than optimum states.

The terms “rolling circle amplification products (RCPs)” and “nanoballs” are intended to have the same meaning and are herein used interchangeably. As used herein, the terms “RCPs”, “concatemeric amplicon products”, and “nanoballs” may not require a condensing agent.

The term “sample” means a source of a target or an analyte. Non-limiting examples of samples include biological samples, such as whole blood, lymphatic fluid, serum, plasma, sweat, tear, saliva, sputum, cerebrospinal fluid, amniotic fluid, seminal fluid, vaginal excretion, serous fluid, synovial fluid, pericardial fluid, peritoneal fluid, pleural fluid, transudates, exudates, cystic fluid, bile, urine, gastric fluid, intestinal fluid, fecal samples, liquids containing single or multiple cells, liquids containing organelles, fluidized tissues, fluidized organisms, liquids containing multi-celled organisms, biological swabs and biological washes. Samples may be from any organism (e.g., prokaryotes, eukaryotes, plants, animals, humans) or other sample (e.g., environmental or forensic samples). “Sample” may mean a set of nucleic acids for testing. A sample preparation process may be used to produce an assay ready sample from a raw sample or partially processed sample. Note that one or more samples may be combined for sample preparation and/or sequencing and may be distinguished post-sequencing using sample-specific DNA barcodes linked to sample fragments.

The term “set” includes sets of one or more elements or objects. A “subset” of a set includes any number of elements or objects from the set, from one up to all of the elements of the set.

The term “subject” includes any plant or animal, including without limitation, humans.

The term “target” means a nucleic acid analyte (e.g., DNA, gDNA, RNA, mRNA, cfDNA etc.) or a proxy for the target analyte of interest (e.g., an antibody conjugated with an oligonucleotide, a cDNA molecule). Thus, in some instances, the term “target” and the term “target analyte” are used interchangeably. “Target” with respect to a nucleic acid includes wild-type and mutated nucleic acid sequences, including for example, point mutations (e.g., substitutions such as single nucleotide polymorphisms, single nucleotide variants insertions and deletions), chromosomal mutations (e.g., inversions, deletions, duplications), and copy number variations (e.g., gene amplifications or gene deletions). “Target” with respect to a nucleic acid may also include the presence or absence of one or more methyl groups on the nucleic acid target. “Target” with respect to a polypeptide includes wild-type and mutated polypeptides of any length, including proteins and peptides.

The term “decoding” with respect to a code includes determining the presence of a known code or a probability of the presence of a known code with or without determining the sequence of the code. Decoding may be hard decision decoding. Decoding may be soft decision decoding.

The term “identify”, “determine”, and the like with respect to codes, targets or analytes are intended to include any or all of: (A) an indication of the presence or absence of the relevant code, target or analyte, (B) an indication of the probability of the presence or absence of the relevant code, target or analyte, and/or (C) quantification of the relevant code, target or analyte.

The terms “hard decision decoding” or “hard decision” refer to a method or model that includes making a call for each nucleotide in a nucleic acid segment (commonly referred to as a “base call”) in order to identify nucleotides in the nucleic acid segment. Models of the inventive concepts incorporate hard decision decoding models. The particular nucleic acid being decoded may be or include a code of the inventive concepts.

The terms “soft decision decoding” or “soft decision” refer to a method or a model that uses data collected during a decoding process to calculate a probability that a particular nucleic acid or nucleic acid segment is present. The probability may be calculated without making a base call for each nucleotide in a nucleic acid segment. In another example, a probability is calculated without making a hard call that a string of nucleic acids in a segment are present. Instead of making a hard call for each nucleotide or nucleotide segment, a probabilistic decoding algorithm is applied to the recorded signal upon completion of signal collection. A probability of the presence of each of the codes may be determined without discarding a signal in contrast to hard decision decoding method in which hard calls are made during the signal collection process. In soft decision decoding, the data may, for example, include or be calculated from, intensity readings in spectral bands for signals produced by the sequencing/decoding chemistry. In one embodiment, soft decision decoding uses data collected during a sequencing/decoding process to calculate a probability that a particular nucleic acid segment from a known set of sequences is present. Models of the inventive concepts may be used for soft decision decoding. The particular nucleic acid or nucleic acid segment being decoded may be or include a code of the inventive concepts.

The terms “phasing” or “signal phasing” refers to misalignment of sequence by synthesis (SBS) cycles during an SBS process caused by the non-incorporation of a nucleotide during a cycle or by the incorporation of two or more nucleotides during an SBS cycle. The term “phasing”, which may also be referred to as “phased sequencing”, may refer to obtaining a sequence and/or alleles associated with one chromosome, or portion thereof, of a diploid or polyploid chromosome. Phased sequencing may capture unique chromosomal content, including mutations that may differ across chromosome copies. In some embodiments, phased sequencing may distinguish between maternally and paternally inherited alleles.

The terms “droop” or “signal droop” means signal decay that occurs during an SBS process, which may be caused by some complementary strands being synthesized as part of the SBS process being blocked, preventing further nucleotide incorporation.

The term “crosstalk” refers to the situation in which a signal from one nucleotide addition reaction may be picked up by multiple channels (referred to as “color crosstalk”) or the situation in which a signal from a nanoball or sequencing cluster interferes with an adjacent or nearby cluster or nanoball (referred to as “cluster crosstalk” or “nanoball crosstalk”).

The term “color channel” means a set of optical elements for sensing and recording an electromagnetic signal from a sequencing or a decoding reaction. Non-limiting examples of optical elements include lenses, filters, mirrors, and cameras.

The terms “spectral band” or “spectral region” means a continuous wavelength range in the electromagnetic spectrum.

Headings are included herein for reference and to aid in locating the various sections. These headings are not intended to limit the scope of the concepts described with respect to the headings.

The description and examples should not be construed as limiting the scope of the inventive concepts to the embodiments and examples described herein, but as encompassing all modifications and alternatives falling within the true scope and spirit of the inventive concepts.

### V. EXAMPLES

The following examples are included for illustrative purposes only and are not intended to limit the scope of the inventive concepts.

### Example 1: Proof of Concept Experiment for Multi-Region Recognition Elements

In this experiment, three coded multivalent recognition elements were designed with a 5′ arm probe that interrogates a genotyping single nucleotide polymorphism (SNP) and a 3′ probe arm that interrogates an anchor SNP with a gap-filling bridge element disposed between them. The three multivalent recognition elements were introduced to three different synthetic target nucleic acid sequences, illustrated in FIG. 6, under conditions sufficient to hybridize the synthetic target nucleic acid sequence to the multivalent recognition elements. Ligation between the 5′ probe arm and 3′ end of the gap-filling bridge element and 5′ end of the gap-filling bridge element and 3′ probe arm was possible when the genotype SNP and/or the anchor SNP were present in the synthetic target nucleic acid sequence and both sequences hybridized to the recognition element complementary sequences (top scenario). Rolling circle amplification (RCA) was performed and fluorescently labeled detection reagents were used to visualize the density, size and uniformity of nanoballs generated in the RCA reaction.

FIG. 6 shows, in the top scenario, that the double ligation between the ends of the multivalent recognition element as the synthetic target nucleic acid sequence hybridized to both the genotyping SNP and the anchor SNP (no mismatches), yielded the highest density, size and uniformity of nanoballs. The middle scenario in FIG. 6 shows a mismatch at the 3′ terminus which resulted in 1% of the multivalent recognition elements being ligated, RCA amplified and detected relative to the top scenario, whereas the bottom scenario, where the mismatch was on the 5′ terminus of the multivalent recognition element resulted in 17% of the multivalent recognition elements being ligated, RCA amplified and detected relative to the top scenario. While mismatches at both 3′ and 5′ ends resulted in a large decrease in the number of detected amplification products, a mismatch on the 3′ terminus resulted in much less off target ligations, amplification and detections when compared to a mismatch being on the 5′ terminus which led to a larger number of off target ligations, amplifications and detections.

Various modifications and variations of the disclosed methods, compositions and uses of the inventive concepts will be apparent to the skilled person without departing from the scope and spirit of the inventive concepts. Although the inventive concepts have been disclosed in connection with specific preferred aspects or embodiments, it should be understood that the inventive concepts as claimed should not be unduly limited to such specific aspects or embodiments.

The present inventive concepts may be implemented using hardware, software, or a combination thereof and may be implemented in one or more computer systems or other processing systems. In one aspect, the inventive concepts are directed toward one or more computer systems capable of carrying out the functionality described herein. Although the foregoing subject matter has been described in some detail by way of illustration and example for purposes of clarity of understanding, it will be understood by those skilled in the art that certain changes and modifications can be practiced within the scope of the appended claims.

While preferred embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

What is claimed is:

1. A method for detecting two or more genomic regions of interest in a target of a set of targets in an assay, the method comprising:
(a) subjecting the set of targets to a hybridization event, wherein each target of the set of targets is uniquely recognized by and hybridized to at least one coded recognition element from a set of coded recognition elements, wherein each coded recognition element comprises:
(i) a first target-specific binding site that flanks a first genomic region of interest binding site to a first of the two or more genomic regions of interest in the target of the set of targets;
(ii) a second target-specific binding site that flanks a second genomic region of interest binding site to a second of the two or more genomic regions of interest in the target of the set of targets; and
(iii) a code from a set of codes, wherein each code from the set of codes comprises at least one segment encoding one or more symbols that correspond to a sequence of one or more nucleotides, and wherein each code from the set of codes is unique for each coded recognition element from the set of coded recognition elements;
(b) subjecting the coded recognition elements from the set of coded recognition elements to a ligation event to yield a set of ligated coded recognition elements; and
(c) performing an amplification reaction on the ligated coded recognition elements; and
(d) detecting the two or more genomic regions of interest associated with the amplified ligated coded recognition elements; wherein the detecting comprises
(e) decoding the codes of the amplified ligated coded recognition elements by recording a signal produced in response to interrogation of each segment of the codes of the amplified ligated recognition elements by one or more hybridization probes, and upon completion of the interrogation determining a probability of the presence of each of the codes of the amplified ligated recognition elements by applying a soft decision probabilistic decoding algorithm to the recorded signal, wherein the presence of the code is indicative of the presence of the target, thereby assaying for the two or more genomic regions of interest in the target of the set of targets.

2. The method of claim 1, further comprising performing (a) to (e) with at least 100 coded recognition elements comprising to predict a presence of a plurality of target nucleic acids.

3. The method of claim 1, further comprising performing (a) to (e) with at least 1,000 coded recognition elements to predict a presence of a plurality of target nucleic acids.

4. The method of claim 1, further comprising introducing a bridge element to the target nucleic acid, wherein the bridge element hybridizes to a region of the target nucleic acid between the first genomic region of interest binding site and the second genomic region of interest binding site, and wherein the ligating in (c) further comprises ligating the bridge element to the 3′ end and 5′ end of the coded recognition element.

5. The method of claim 1, wherein the amplification reaction comprises performing rolling circle amplification to generate concatemeric amplification products.

6. The method of claim 5, further comprising:
(a) cleaving the concatemeric amplification products to yield a plurality of unit length monomer fragments each comprising a copy of the code;
(b) re-circularizing the unit length monomer fragments to generate re-circularized monomers; and
(c) amplifying the re-circularized monomers in a second rolling circle amplification reaction to produce multiple rolling circle amplification products of the re-circularized monomers.

7. The method of claim 1, further comprising subjecting the amplified coded recognition elements to an exonuclease reaction.

8. The method of claim 1, wherein the presence of the target nucleic acid is informative for:
(a) pathogen detection;
(b) leveraging variable regions within pseudogenes to disambiguate a genotype;
(c) identifying methylation events from bisulfite converted DNA or from non-treated samples; or
(d) any combination of (a), (b) and (c).

9. The method of claim 1, wherein each code comprises four to sixteen segments.

10. The method of claim 1, wherein at least one of the segments is interrogated more than one time by hybridization with one or more hybridization probes, wherein the one or more hybridization probes comprises an optical label or a fluorescent label.

11. The method of claim 10, wherein the one or more hybridization probes comprises one optical label of at least four different optical labels or one fluorescent label of at least four different fluorescent labels.

12. The method of claim 1, further comprising performing (a) to (e) for hundreds of targets comprising said target.

13. The method of claim 1, wherein the two or more genomic regions of interest comprise two or more point mutations, two or more substitutions, two or more insertions, two or more deletions, two or more copy number variations, or any combination thereof.

14. The method of claim 1, wherein each code from the set of codes is a predetermined code based, at least in part, to avoid assay component interactions; and wherein the code is homopolymer free.

15. A set of multi-region coded recognition elements, wherein a multi-region coded recognition element of the set of multi-region coded recognition elements comprises:
(a) a first target-specific binding site that flanks a first genomic region of interest binding site;
(b) a second target-specific binding site that flanks a second genomic region of interest binding site; and
(c) a code from a set of codes, wherein the code is a soft decodable code comprising at least one segment encoding one or more symbols that correspond to a sequence of one or more nucleotides.

16. The set of multi-region coded recognition elements of claim 15, wherein the set of multi-region coded recognition elements are padlock probes or molecular inversion probes.

17. The set of multi-region coded recognition elements of claim 15, further comprising a bridge element that, when bound to a target, is disposed between the first genomic region of interest binding site and the second genomic region of interest binding site of the multi-region coded recognition element.

18. The set of multi-region coded recognition elements of claim 15, wherein the set of multi-region coded recognition elements comprises at least 10 multi-region coded recognition elements.

19. The set of multi-region coded recognition elements of claim 15, wherein the set of multi-region coded recognition elements comprises at least 100 multi-region coded recognition elements.

20. The set of multi-region coded recognition elements of claim 17, wherein the multi-region coded recognition elements in the set of multi-region coded recognition elements are hybridized to the first target specific binding site, the first genomic region of interest, the second target specific binding site, and the second genomic region of interest of a target of interest and comprises a contiguous nucleic acid molecule as a result of a ligation or gap-filing ligation of a 5′ probe arm of the multi-region coded recognition elements, the bridge element, and a 3′ probe arm of each of the multi-region coded recognition elements.
